(19) United States
(12) Patent Application Publication
(10) Pub. No.: US 2007/0225213 A1
(43) Pub. Date: Sep. 27, 2007

(54) Nucleic acid carriers for delivery of therapeutic agents

(76) Inventor: Matthew K. Kosak, Salt Lake City, UT (US)

Correspondence Address: Matthew K. Kosak, 272E. New Century Ln #F, Salt Lake City, UT, 84115, US

(21) Appl. No.: 11/387,197

(22) Filed: Mar. 23, 2006

(51) Int. Cl. A61K 48/00; A61K 31/704; A61K 31/337; A61K 31/282; A61K 31/525; A61K 33/24

(52) U.S. Cl. 514/7; 514/44; 514/34; 424/649; 514/251; 514/11; 514/492; 514/449

(57) ABSTRACT

Nucleic acid drug carriers comprise a nucleic acid carrier complexed with a drug, wherein the nucleic acid carrier and the drug are associated non-covalently, and optionally other agents such as spacer, transfection agents, and targeting agents. The nucleic acid drug complex are discovered to have permissive or refractory uptake depending on many factors including cell type, proliferation rate, among others. The refractive uptake of the nucleic acid drug complex are shown to be useful in the nucleic acid targeting of drugs, both in vitro and in vivo. Novel drug compositions are disclosed that effectively reduce the toxicity of drugs while maintaining drug activity and enhancing a drug's therapeutic index.

### TECHNICAL FIELD OF THE INVENTION

This invention relates to the discovery of nucleic acid carriers that facilitate nucleic acid targeting of therapeutic agents in a drug delivery system. More specifically, this invention relates to the combination of a nucleic acid carrier with a therapeutic agent to form a novel therapeutic entity, herein referred to as a nucleic acid-drug complex or NAC-drug. Nucleic acid carriers are found to reversibly bind and inactivate a drug, thereby facilitating drug co-transport and masking. The nucleic acid drug complex is discovered to have cell specific targeting based on factors that pre-dispose cells for uptake of the nucleic acid drug complex. In other aspects, the invention utilizes the refractive nature of cells toward the nucleic acid-drug complex in order to enhance in-vivo distribution and drug delivery to a target. By selection of appropriate nucleic acid-drug system, the nucleic acid carriers can directly address the current need for delivering drugs safely to their desired destination with reduced side effects to the body. The NAC-drug complex is discovered to have novel, bio-pharmaceutical properties and offers new alternatives for the treatment of cancer and other diseases.

### BACKGROUND OF THE INVENTION

Nucleic acid is perhaps one of the most important molecules known. Since its structural elucidation in 1953, the applications of nucleic acids have grown significantly and now represent a new field of bio-chemical engineering. Recently, DNA-based therapeutics have been proposed in the treatment of many types of human illness. For example, antigene therapeutics are proposed that elicit cellular effects by highly specific, Watson-Crick hybridization to a cellular mRNA or genomic DNA target. Although significant work is underway in the use of DNA-based technologies as an experimental tool for the study of gene silencing and replacement, problems of efficacy, as well as delivery, have limited the progress of nucleic acids in clinical applications.

On the other hand, small drugs still offer real clinical benefits in the treatment of a broad range of human disorders. Advancements in drug discovery have produced large libraries of bio-active compounds that are highly potent. However, despite these gains, a significant need still exists for therapeutics that are more potent against a target and yet have fewer side-effects to the patient. Consequently, drug carriers that enhance solubility, bioavailability, efficacy, safety, and targeting of therapeutic agents are continuously needed.

One of the current strategies for drug delivery involves pro-drugs. In pro-drug conjugates, such as HPMA-doxorubicin, the therapeutic agent is conjugated to a hydrophilic polymer via a biodegradable or acid labile spacer arm. For pro-drugs to be effective, the spacer must resist degradation in the blood stream while at the same time releasing the free drug in the cellular lysosome. Pro-drug approaches have problems with release but also targeting. Because conjugation typically only anchors the drug to the carrier molecule, the conjugation step can ineffectively mask drug activity, leading to toxicity. The permissive uptake of pro-drug conjugates into healthy cells as well as cancer cells is also a problem, and can cause dose-limiting toxicity and reduce the therapeutic index of pro-drugs. Finally, the introduction of novel polymeric carriers into the body that have unknown metabolism or bi-products, can further reduce the advantages and increase the potential for rejection.

Nucleic acids are poorly internalized by eukaryotic cells. The poor uptake of nucleic acids by cells is likely a defense mechanism evolved against viruses and ensures the integrity of the cellular genome against entry by foreign DNA and RNA. Their use as drug carriers is therefore challenged by the cells natural tendency to repel foreign DNA's, their polyanionic nature, size, poor endosomal release, and instability and degradability by enzymes. To use DNA as an effective carrier of drugs, these hurdles must be overcome. However, as shown herein, it is the unique features and challenges of DNA which make it attractive as a drug delivery molecule.

In oncology, there is currently a critical need for targeted therapeutics. Anti-neoplastic drugs are currently the best treatments for many types of cancers which are inoperable or refractory to other treatments. However, anti-neoplastic drugs are limited by severe side-effects and dose-limiting toxicity which reduce their therapeutic index. In addition, the lack of targeting and dose-limiting toxicity contributes to tumor resistance since inadequate concentrations of drug reach the tumor. Pro-drug formulations as well as liposomes and micelles, have attempted to address masking and targeting of small drugs, but have been slow to yield clinically acceptable alternatives.

Effective targeting is needed to increase the drug concentration in solid tumors, enhancing lethality to the cancer, and minimizing the chance for drug resistance. The preferential, targeted delivery of cytotoxic agents to rapidly growing cancer cells would represent a breakthrough in cancer treatment and provide a new avenue for therapeutic options.

In neoplasia, cells have undergone a complex process of transformation, induced either by a virus, a chemical agent, radiation, genetics, or a combination of factors. Although most if not all cancers are characterized by a transformed state, tumorogenesis is a final state in the transformation process, in which the cell growth is uncontrolled.

It is hypothesized that transformed and neoplastic cells have a greater tendency for the uptake of nucleic acids and so can be targetable by the nucleic acid-drug carrier. In-vitro results support the model and show significant, preferential uptake of the NAC's in fast growing, neoplastic cells as opposed to primary cell lines.

The potential of the present compositions for offering a new approach and therapeutic alternative in the treatment of cancer and other diseases is therefore significant. Because the nucleic acid-drug complex is a macromolecular entity, it offers a unique pathway of entry of the small drug into the cell, likely by active pinocytosis and endocytosis events rather than simple diffusion. The high biocompatibility of the nucleic acid carrier also has many advantages. For example, in-vitro results show that nucleic acid carriers exhibit high drug loading while at the same time providing effective masking of drug toxicity. The invention can provide nucleic acid targeting for therapeutic agents, based on cellular factors, that increase or decrease accumulation of nucleic acid drug complex in a cell. For example, in studies aimed at exploring the function of nucleic acid drug carriers, it was discovered that accumulation of drug can be enhanced in rapidly proliferating cells and in non-established cells. Since tumor cells are typically the major proliferating cells in the adult, these cells can accumulate high concentrations of NAC-drug in preference to other tissues.

Nucleic acid carriers (NAC's) can deliver and release active agents to a sub-cellular location by a series of novel mechanisms. First, NAC's are uptaken primarily into cells that are porous to the DNA-drug complex. Next, after uptake of NAC-drug complex, the drug is released within the cell, via lysosomal transport to the nucleus or by degradation of the carrier by intracellular enzymes. In cell culture, nucleic acid-drug carriers are discovered to selectively enter non-established cells in preference to established lines. The nucleic acid targeting of small drugs can be influenced by intracellular factors, phenotype, metabolic activity, and rate of division, factors that can increase or reduce cellular uptake. The factors involved in the uptake and intracellular trafficking of the nucleic acid-drug are complex, and it is possible that the nucleic acid and drug act in unison to influence cellular function.

Significant therapeutic benefit can be obtained from employment of the present carriers in the formulation of many small molecular drugs. One example of a class of drugs which can significantly benefit from formulation in a NAC are drugs which target the cellular DNA and RNA contained within the nucleus and the surrounding cytoplasm. DNA targeting, cytotoxic agents work by interfering with enzymes which are necessary for DNA function and replication. DNA targeting, cytotoxic drugs work by a variety of mechanisms including minor groove binding, covalent binding (alkylation), and strand cutting. Cytotoxic drugs which target DNA, comprise a significant number of drugs currently used against cancer, including anthracycline antibiotics, platinum intercalators (cisplatin), nitrogen mustards (DNA alkylating agents), and synthetic intercalators (anthracene based). However, because DNA targeting drugs are non-discriminatory, and broadly acting, they can produce systemic toxicity and severe side effects.

Other therapeutic avenues are possible by combining the nucleic acid carrier with anti-mitotic agents such as paclitaxel and docetaxel, which bind the microtubule protein, tubulin. Paclitaxel and related taxanes can be formulated with the nucleic acid carrier to provide numerous benefits such as enhanced solubility, reduced side-effects, and nucleic acid-paclitaxel targeting.

The unique targeting aspects of the present invention can be utilized in the treatment of viral infections, since viruses can potentially alter a cells porosity to the nucleic acid-drug carrier. Oncogenic viruses are known to cause cellular transformation and can render these cells susceptible to preferential uptake of the nucleic acid-drug complex, causing differential uptake of drug in these cells and greater cell morbidity. Targeting and eliminating the host cell with the nucleic acid-drug, rather than directly attacking the virus, can be potentially efficacious against certain viral infections.

Gene based therapies such as anti-sense, antigene, and plasmids have been proposed in the treatment of certain viral diseases and cancer. Recently, it has been proposed to use anti-sense oligonucleotides (ODN) to treat cancer by down regulating a gene that confers resistance by cancer to apoptosis. Antisense and antigene oligonucleotides have been proposed that bind to a complimentary mRNA target or genomic DNA target strand within the cell, causing cleavage of the message, and reduction in the protein. However, the utility of antigene and antisense therapeutics have been limited by many factors such as 1) low cellular uptake, 2)degradation by nucleases, and 3) escape from intracellular compartments. Ineffective uptake leads to a sub-optimal concentration of the antisense therapeutic in the cell to hybridize with the mRNA and silence the protein, and protein is rapidly restored. Furthermore, the lack of efficacious delivery system has required high doses of oligonucleotides leading to serious side effects such as thrombocytopenia. Antigene and antisense therapeutics have proven impractical for the treatment of cancer, even when used in conjunction with existing treatments. In recent phaseill clinical trials against cancer, antigene therapy used in conjunction with current treatments, such as dacarbazine, have shown higher toxicity than with dacarbazine alone, and their higher toxicity and risk has outweighed their potential clinical benefit. In cancer treatment, the downregulation of a protein target by antigene or antisense mechanisms has lacked potency, either due to problems of mechanism or lack of sustained silencing in-vivo. On the other hand, NAC-drugs offer a novel, alternative approach.

Unlike current gene-based therapies, nucleic acid-drug carriers operate primarily against small drug targets. This novel approach produces a drug entity that has the potency and ruggedness of a small drug, but without the drawbacks of small drugs, namely their non-selectivity. Furthermore, as demonstrated herein, nucleic acid-drug carriers overcome the problems of intracellular delivery and efficacy encountered by current gene- based drugs, due in part, to their more robust mechanism of action. Unlike current gene- based therapies, nucleic acid-drug carriers can operate independently of sequence specific hybridization of the nucleic acid to a cellular mRNA target. Instead, nucleic acid drug carriers mediate their cellular effects primarily by delivery of the active drug to a sub-cellular target, such as cellular DNA or a protein. As a result, nucleic acid drug carriers can more broadly and effectively target cellular processes that lead to cell apoptosis, among others, by targeted delivery of highly potent anti-mitotic drugs, such as paclitaxel. Furthermore, despite the intrinsically low uptake of nucleic acids, nucleic acid-drug carriers are discovered to have significant uptake in a broad range of cultured cells and in the absence of transfection agents.

Another significant problem in cancer treatment is multiple drug resistance (MDR). Multiple-drug resistance is believed to occur based on the over expression of proteins (p-glycoproteins) in the cellular membrane that pump small drugs out of the cytoplasm. Nucleic acid-drugs can address this problem by changing the pathway of drug uptake. Since nucleic acid-drugs are transported via lysosomal membranes, they can by-pass the cytoplasm and can maintain an effective concentration in the cancer cells.

A current need exists for novel therapeutic agents that can increase treatment success and efficacy but at the same time reduce side-effects to the patient. New approaches are required in the treatment of cancer, which is currently a devastating disease. The present invention shows for the first time, the utility of nucleic acid carriers for the delivery of drugs, nucleic acid targeting of drugs, and their potential for therapeutic applications. Methods and compositions for the novel encapsulation of drugs by nucleic acids, their effective cellular targeting and subsequent release of active agents are disclosed.

### SUMMARY OF THE INVENTION

The invention provides novel compositions and methods for the use of nucleic acid carriers that provide nucleic acid targeting, masking, release, and delivery of therapeutic agents to a location within a cell or the body. The invention comprises a drug and a nucleic acid carrier with one or more drug binding sites. In other embodiments, the invention further comprises masking agents, transfection agents, and targeting moieties. The principal elements of the invention are briefly summarized in the following:

1) The invention utilizes a drug-targeted, nucleic acid carrier to form a complex with a drug, and optionally, to reversibly bind and inactivate a drug.

2) The invention further provides nucleic acid targeting to drugs by incorporation of a nucleic acid binding spacer (NABS). The NABS can comprise an intercalator, minor groove binder, DNA intercalator, a peptide, heterocyclic residue, or other suitable agent to provide suitable non-covalent association between the drug and the NAC carrier.

3) The invention optionally comprises one or more neutral masking agents. Suitable masking agents include cholesterol, lipid, polysaccharide, polyethylene glycol, or fatty acid.

4) The invention can include one or more transfection agents to enhance uptake and nucleic acid targeting.

5) Finally, the invention optionally comprises one or more targeting moieties, grafted to the NAC.

The nucleic acid-drug carrier can be administered in a dosage form, such that the drug is substantially bound and free drug is substantially minimized. Applications for NAC-drug compositions include research as well as pharmaceutical applications which are suitable for human or veterinary medicine.

The invention and its attendant advantages can be more readily appreciated by reference to the following figures.

### BRIEF DESCRIPTION OF THE DRAWINGS

FIG. 1. Shows the effect of drug loading on toxicity of NAC-MTN or NAC-DOX against MCF7 breast cancer cells. NAC-MTN or NAC-DOX were prepared at different weight:weight % of drug:nucleic acid and incubated in cells. For each sample and corresponding % loading, the total drug was held constant at 0.058 mM MTN or 0.1 mM DOX. After incubation in cells, cell viability was assayed via mitochondrial dehydrogenase activity against 3-[4,5-dimethylthiazol-2-yl]-2,5-diphenyl tetrazolium bromide (MTT) and reported as % toxicity relative to untreated cells. Higher drug loading was shown to cause more toxicity correlating to greater presence of free drug.

FIG. 2. The binding of DNA with dipyridamole (EX081205) is shown as an increase in dipyridamole fluorescence. Dipyridamole (DPA) was complexed with varying amounts of nucleic acid in water and the fluorescence taken on Synergy HT1 (BioTech Inc).

FIG. 3. Solubility enhancement of paclitaxel by NAC. Aqueous paclitaxel solutions were prepared with or without DNA, sonicated, stored at room temperature for several hours, and then centrifuged. The pellets were resuspended in 50% ethanol and the precipitated paclitaxel was quantified using a standard absorbance curve at 230 nm. The total percentage of solubilized paclitaxel was calculated using %=100× (1-assy mM)/expected mM.

FIG. 4. Shows the differential toxicity of NAC-MTN in faster growing cell lines. The relative toxicity of NAC-MTN was compared with MTN and CD-MTN in four cell lines, MCF7(breast cancer), COS7green monkey kidney (SV40 transformed), 3T3 (mouse fibroblast), and BHK21(hamster kidney). Cells were treated with identical amounts of MTN formulated as either NAC-MTN, MTN only, or poly-beta-cyclodextrin-sulfylbutylether(CD-MTN). After an incubation period, cell viability was assayed with MTT assay. The differences in toxicity between NAC-MTN and MTN or NAC-MTN and CD-MTN are shown as % difference. Greater percentage difference correlates to more reduction in relative NAC-MTN toxicity. Lower percentage difference indicates more equivalent toxicity (data from table in example 10). The differential targeting of NAC-MTN is demonstrated in slower proliferating lines MCF7 and 3T3 than in COS7 and BHK21.

FIG. 5. Assay for free drug in nucleic acid carrier-mitoxantrone (NAC-MTN, EX050404). MCF7 cells were treated with NAC-MTN with different drug loading formulations. The total MTN is shown for MTN only and NAC-MTN. Cells were treated with different amounts of NAC-MTN, at either 5%, 10% and 20% weight:weight of MTN to HSDNA (<or=100 bp), after incubation with MTN or NAC-MTN, cell viability was assayed via mitochondrial dehydrogenase activity against 3-[4,5-dimethylthiazol-2-yl]-2,5-diphenyl tetrazolium bromide (MTT). Surprisingly, at the highest drug loading, or 20%, the toxicity was lowest. The free MTN curve shows toxicity to MCF7's without the carrier. At 20% loading, the NAC-MTN produces toxicity equivalent to MTN at only at 5 ug/ml, or about a 30 fold reduction. Based on the assay, free drug in NAC-MTN at 20% loading is less than or equal to about 3%.

FIG. 6. Assay of free drug in NAC-DOX by cell culture. Breast cancer cells were treated with differing amounts of NAC-DOX, at 8% fixed drug loading, to give 0.008, 0.016, 0.03, or 0.06 mg/ml final DOX. After incubation in cells, cell viability was assayed by MTT assay and expressed as % toxicity (data from example 5).

FIG. 7. Comparing MCF7 toxicity of NAC-DOX and NAC-DOX delivered with pPEG-PEI. Nucleic acid-DOX carriers were prepared by complexing degraded herring sperm DNA (<or=100 bp, HSDNA) with doxorubicin HCl in H20 followed by dilution in 1× DMEM. MCF7 cells were treated with differing amounts of nucleic acid-DOX carrier, with and without complexation with PEG shielded micelle, and assayed for cell viability using MTT assay. Nucleic acid-DOX carrier delivered with the PEG shielded micelle (HSDNA-DOX_pPEG1020H) shows significantly higher toxicity than nucleic acid DOX alone (HSDNA-DOX).

FIG. 8. Differential targeting and distribution of NAC-MTN or MTN in major organs of the mouse. NAC-MTN or MTN was administered intravenously to mice and detected in major organs after 24 hours. Total effective MTN dosage for each mouse was 0.62 mg MTN per mouse. Total MTN in tissues was quantified by MTN fluorescence, normalized against total tissue weight, and reported as MTN mg/g tissue. Mice treated with MTN alone, showed more than two-fold higher concentrations of MTN in the heart, lung, and liver than mice treated with NAC-MTN. In the kidneys, MTN was equivalent for NAC-MTN and MTN at 0.26 mgMTN/g tissue (data from example 17).

FIG. 9. Differential clearance of NAC-MTN and MTN from the circulation (EX110105). NAC-MTN or MTN alone was administered intravenously to mice via the tail vein. The effective MTN dosage for each mouse was 0.28 mg. After 2 hours, NAC-MTN or MTN was detected in urine, quantified by absorbance at 665 nm, and reported as % of total. Clearance of MTN from blood in NAC-MTN treated mouse was over four-fold higher than in MTN treated mouse.

FIG. 10. Shows the binding of nucleoside (azidothymidine, AZT) with DAPI and deoxynucleoside tri-phosphates of adenosine, cytidine, guanidine, thymidine to DAPI, mitoxantrone (MTN), doxorubicin (DOX), and DAPI by fluorescence. The fluorescence of each complex was taken at EX360/460, EX620/680, or EX485/528 and plotted against nucleoside concentration.

FIG. 11. Complexation of deoxynucleoside triphosphate with DNA in the presence of DAPI is shown as enhanced fluorescence. Enhanced fluorescence of HSDNA-DAPI-dNTP complex was measured by subtracting fluorescence of HSDNA-DAPI from HSDNA-DAPI-dNTP. The increase in fluorescence is shown to be proportional to the DNA concentration.

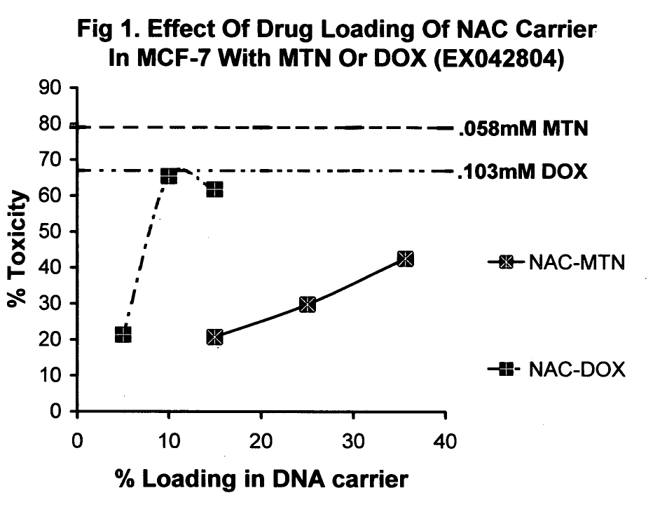

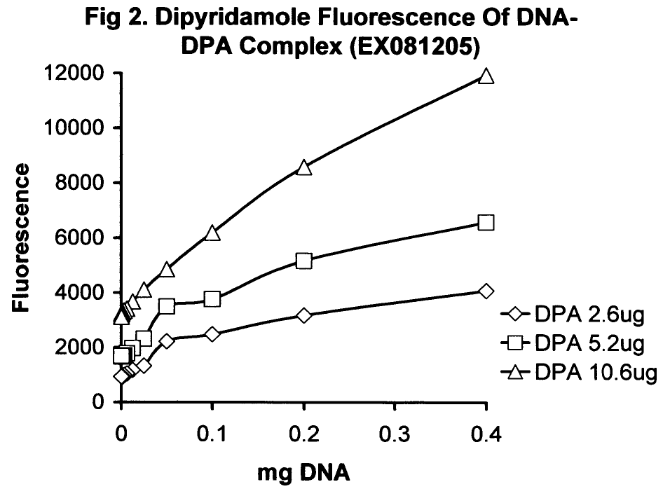

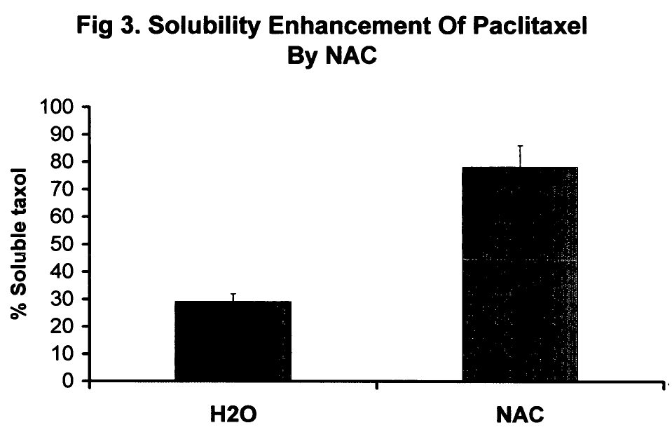

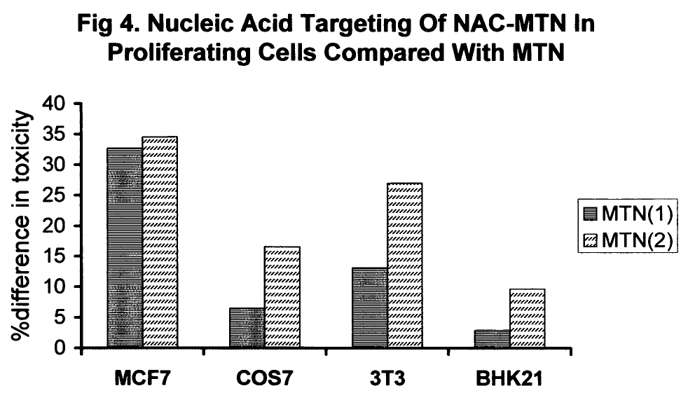

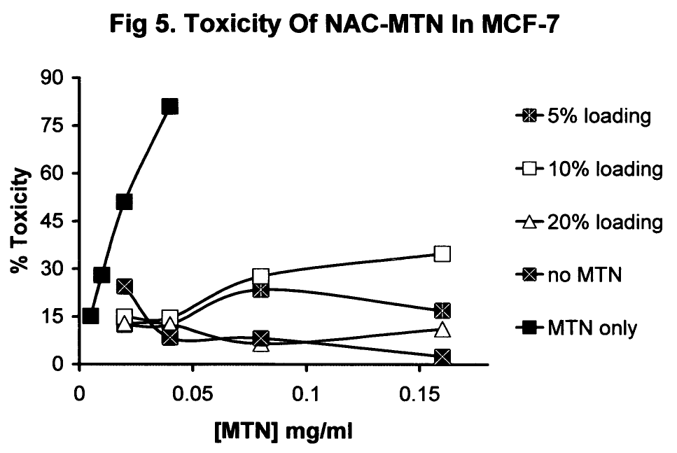

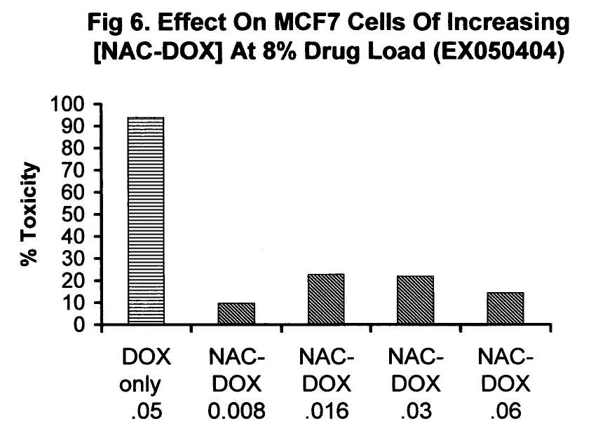

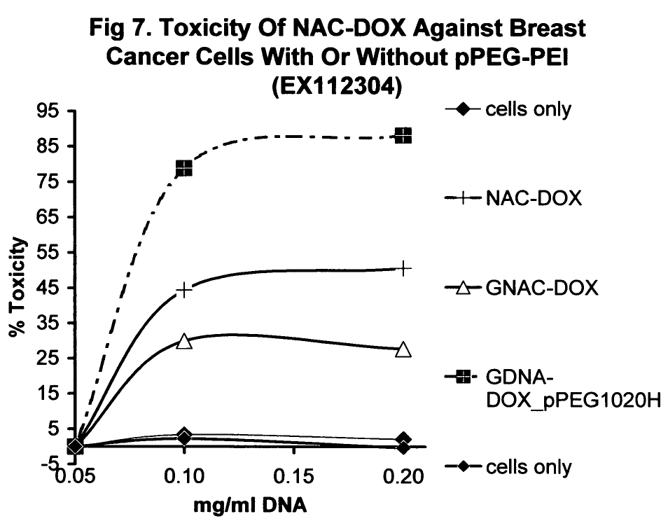

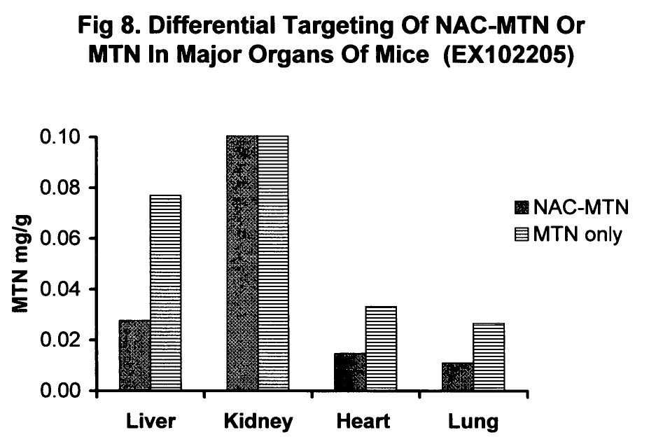

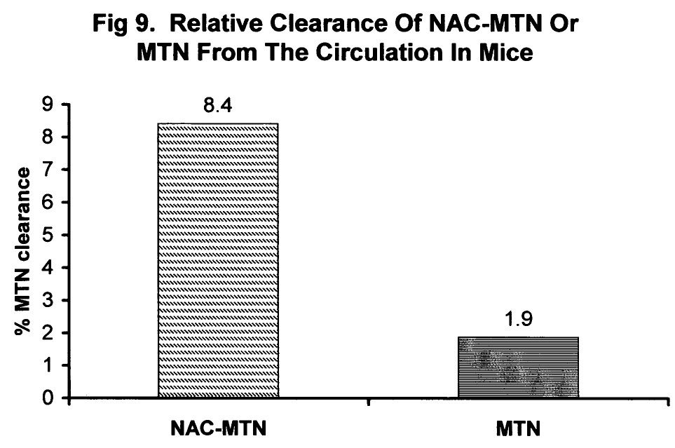

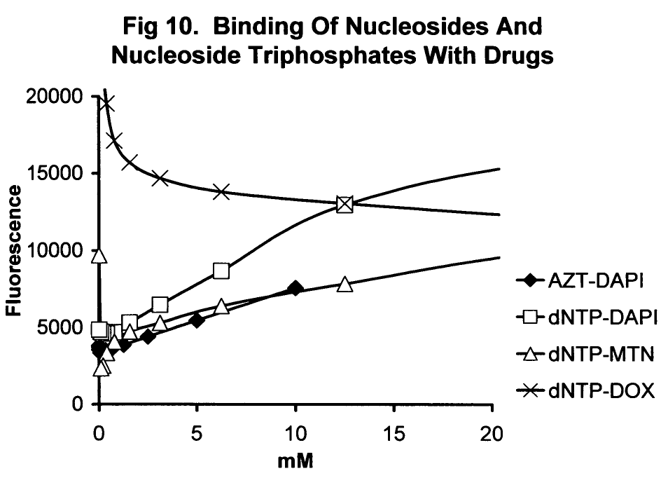

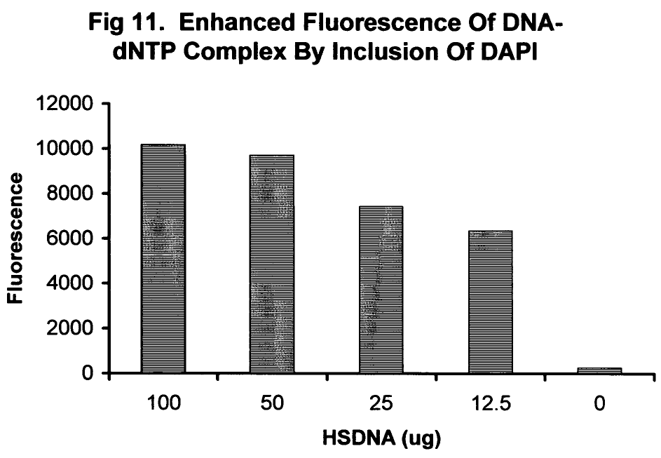

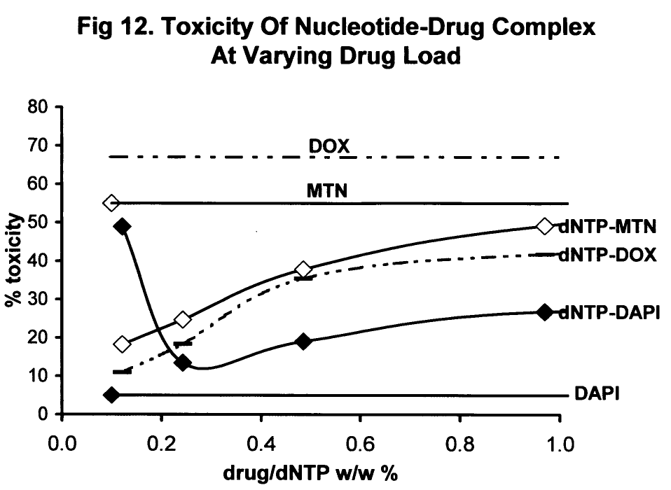

### DESCRIPTION OF THE PREFERRED EMBODIMENTS

For the purposes of disclosing this invention, certain words, phrases and terms used herein are defined below. It is to be understood that this invention is not limited to the particular configurations, mechanisms, or processes described as such configurations, mechanisms, or processes may vary somewhat. It is also to be understood that the terminology employed is only for describing the particular embodiments and is not intended to limit the invention.

Drug

“Drug” and “therapeutic agent” herein refers to a bio-active molecule that has useful therapeutic or pharmaceutical activity and is generally acceptable for use in human or veterinary medicine. The term also includes any bio-actives that have potential utility as drugs. The “drug” herein also includes conjugated forms of the bioactive agent. Unless otherwise specified, the term “drug” or “therapeutic agent” as used herein, refers to the drug entity, payload, bio-active that is associated with the nucleic acid. Unless otherwise specified, the term “drug” herein most often refers to organic drugs and small drugs of less than 10000 mw but more typically less than 5000 mw. If more than one entity in the nucleic acid-drug composition is bio-active, the term “drug” or “therapeutic agent” more preferably refers to the primary, bio-active agent in the composition. Useful drugs and classes of drugs that have utility in the present invention can be found in the Physician's Desk Reference (PDR) or MERCK Index and include, but are not limited to, nucleic acid binding drugs, intercalator drugs, minor groove binders, anti-mitotic drugs, anti-inflammatory drugs, alkaloids, peptide drugs, nucleoside drugs, alkylators, protease inhibitors, antibiotics, antifungals, protein drugs, and drug conjugates.

DNA Intercalating Drug

DNA intercalating drugs are herein defined as bio-active agents that have the capacity to interact with nucleic acid by intercalation. DNA intercalating drugs include doxorubicin (DOX), adriamicin, daunomicyn, and mitoxantrone (MTN), anthraquinonone antibiotics, actinomycins (from streptomyces), anthracenediones, anthraquinone derivatives, acridines, porphyn's and derivatives. Other drugs that intercalate DNA and have utility as therapeutic agents are also included.

Groove Binding Drug

A drug that binds to the minor or major groove of DNA. Groove binding drugs include berenil, netropsin, and distamycin.

DNA Alkylating Agents

These include agents which covalently bind to DNA such as mechlorethamine, otherwise known as nitrogen mustards. Other nitrogen mustards are cyclophosphamide and chlorambucil. Mechlorethamine can selectively crosslink guanine bases of DNA. Other drugs include calicheamicin and mitomycin C (Vrudhula et al. J. Med. Chem. 40:2788 (1997)]. Platinum intercalators such as cisplatin and carboplatin, can also form DNA adducts within sequential guanine bases.

Nucleic Acid Binding Spacer (NABS)

Association between the drug and the nucleic acid can be enhanced by conjugation of the drug to a nucleic acid binding spacer (NABS) to form a drug conjugate. The nucleic acid binding spacer is preferably selected from a compound that intercalates DNA or binds to the groove of DNA, or interacts by hydrogen bonding, charge, or a combination thereof. The NABS is most preferably selected from a nucleic acid intercalator, nucleic acid binding drug, groove binder, nucleic acid binding peptide, or nucleic acid binding peptide fragment. In preferred embodiments, the NABS can serve primarily to non-covalently link various active drugs to the NAC. Various nucleic acid binding compounds that are not necessarily efficacious as drugs, but are highly suitable as NABS, can be useful in the invention to associate effectively and simultaneously with both the nucleic acid and the drug to form novel NAC-drug complexes. Other suitable NABS include, but are not limited to AT selective bis-(6-chloro-2-methoxy-9-acridinyl)spermine, 9-amino-6-chloro-2-methoxyacridine, 4′, 6 diamidino-2-phenylindole (DAPI), SYBR™dyes and derivatives (Invitrogen Inc. CA), acridine orange, minor groove binding Hoechst dyes, GC selective 7-aminoactinomycin D, hydroxystilbamidine, cyanine dyes, cyanine dimers, riboflavin, riboflavin, N-hydroxy succinimide derivatives, thiazole orange, trioxsalen, psoralen, among others.

Nucleic Acid Chimeras

“Nucleic acid chimeras” are obtained by grafting nucleic acid bases (i.e. adenine, cytosine, guanine, and thymine) to a suitable backbone such as a PEG, polysaccarhide, dendrimer, polyglutamate, dextran, protein, or a peptide (i.e. PNA), such that the nucleic acid bases are exposed to the polymer surface, preferably such that said nucleic acid bases are accessible to form useful associations with the drug moiety.

Peptide Drug

Peptide drugs such as granulocyte colony stimulating factor (GCSF, Neulasta ™), PEG-α-interferon, have been recently approved. Also included are peptide intercalators. Highly preferred are pentapeptide agents such as dolastatin 10, and synthetic analogues of dolastins, auristatin E (AE) and monomethylauristatin E (MMAE) disclosed by Pettit et al [Fortschr.Chem.Org.Naturst. 70:1-79 (1997)] and Doronina et al. [Nat. Biotech. 21:778-784 (2003)], that inhibit tubulin polymerization. Also included are antibody drugs such as Mylotarg™ (mAB-calicheamicin), edrecolomab (colon cancer), trastuzamab (breast cancer), rituximab (chimeric antibody against B-cell surface antigen).

Nucleic Acid Carrier

The “nucleic acid carrier” or NAC, is preferably selected from a suitable nucleic acid, modified nucleic acid, phosphorothioate nucleic acid, phosphodiester nucleic acid, alkyl phosphonate nucleic acid, nucleic acid mimetic, nucleic acid co-polymer, nucleic acid chimera, nucleic acid fragment, or any combination thereof. Suitable nucleic acid carriers include any nucleic acids conjugated to suitable masking agents, i.e. polyethylene glycol, polyglutamic acid, cyclodextrin, dextran, human serum protein, cholesterol, vitamins, antibodies, antibody fragments, among others. In other preferred embodiments the NAC is selected from any monomeric forms of nucleic acids, including nucleosides, nucleotides, nucleoside conjugates, or nucleoside analogues, since nucleosides and nucleotides are also discovered to advantageously bind and mask the toxicity of drugs. Suitable nucleosides include deoxynucleosides, dideoxynucleosides, nucleoside diphosphates, nucleoside tri-phosphates, nucleoside dimers, nucleoside trimers, nucleoside fatty acid conjugates, nucleoside cholesterol conjugates, among others. More preferably, the NAC is suitable in forming said complex with the drug, such that the nucleic acid carrier facilitates the co-transport or co-migration of the drug with the NAC.

Nucleic Acid Targeting

Nucleic acid targeting encompasses, but is not limited to, all processes by which the nucleic acid-drug complex selectively enters or targets cells, described herein, based on a cells porosity or refractivity toward a nucleic acid-drug carrier. The nucleic acid targeting aspects also refer to elements in the composition that influence tissue specific uptake. The cells capacity for uptake of nucleic acid is controlled by a set of complex factors including phenotype, transformation, phase, rate of division, pinocytosis, endocytosis, metabolism, cell receptors, and intracellular trafficking. Moreover, the nucleic acid targeting refers herein more generally to the action of nucleic acid-drug carriers, which relate to the complex processes of selective uptake, but also to novel mechanisms of intracellular trafficking and release.

Drug Masking

“Drug masking” is defined as a process that reduces or limits the binding, chemical reactivity, or function of a drug. More preferably, the masking effect is reversible, such that the activity of the drug is not compromised once the drug reaches the target. The drug masking of the nucleic acid-drug complex is determined by assay of drug activity, typically with a cell culture assay, utilizing a cell line that is appropriate for the drug. Since drug masking is dependent on binding of the nucleic acid to the drug, it is also predictive of the suitability of the nucleic acid against the drug and of the drug loading, since both effect cell toxicity. For cytotoxic drugs, the drug masking of the nucleic acid-drug carrier is defined herein as a reduction in toxicity of between 1% and 99.9%, as compared with free drug, and more preferably between 2.5% and 99.9% as determined by MTT cell viability assay. For other drugs the drug masking is determined as a reduction in drug activity.

Liposome

“Liposomes” are small vesicles capable of entrapping drugs within their aqueous interior. Lipsomes are composed of spherical or concentric bi-layers of amphipathic lipids and are typically classified as small unilamellar (SUV), large unilamellar (LUV), or multi-lamellar vesicles (MLV).

Complexing

Without limiting the invention to a specific mode of action, the terms “complexing”, “binding” and “encapsulation” are used synonymously in the present invention to refer primarily to the non-covalent association between the nucleic acid and the drug.

Nucleic Acid Drug Complex

The terms “nucleic acid-drug complex”, “nucleic acid carrier-drug complex”, and “nucleic acid drug carrier” refer herein to the complex produced by associations between the drug and the nucleic acid carrier. The association is preferably non-covalent. Non-covalent associations between the NAC and drug include but are not limited to, intercalation, minor groove binding, charge-charge interaction, hydrogen bonding, Watson-Crick hybridization, aptamer binding, aptamer folding, nucleic acid ligand binding, among others, and can be characterized by a dissociation constant. The percentage of drug that is in a bound state with the NAC is related to the dissociation constant of the NAC-drug complex and is calculated as % molar fraction (mol/mol) or weight fraction (w/w) of total drug present, e.g. % bound drug=100 × (bound drug)/(total drug). The fraction of bound drug in the composition can be determined by fluorescence, absorbance, column chromatography or other means known in the art. It is preferred that the percentage of bound drug ranges from 0.01% and 100%, but more preferably between 0.1% and 100% , and still more preferably between 5% and 100%.

Drug Free Composition

A “drug free” composition is substantially composed of nucleic acid-drug in complexed form with minimal free drug. In some cases, these compositions can be purified to minimize unbound drug or they can be prepared at an optimal nucleic acid-drug ratio, or nucleic acid-drug mass loading ratio, determined empirically for the composition. A preferred drug free composition exists at the optimal ratio of nucleic acid to drug which maximizes the concentration of nucleic acid-drug complex and minimizes the concentration of free drug in solution at equilibrium.

Conjugation

“Conjugation” (or “grafting”) is understood in the art as the process of chemically linking agents with a covalent bond.

Covalent bond

“Covalent bond” refers to single, double, or triple bonds created by the sharing of electrons between atoms, as understood and defined in the art.

Non-covalent

“Non-covalent” refers to associations between atoms and molecules such as ionic, hydrogen bonding, dipole-dipole, non-polar, among others.

Encapsulation

Encapsulation is used to describe non-covalent guest molecule and host molecule associations that cause one molecule to be entrapped or encapsulated within another molecule, such that it can be co-transported with the host molecule. Examples of host molecules include cyclodextrins and dendrimer boxes.

Nucleic Acid

The nucleic acid is preferably a deoxyribonucleic acid (DNA) or ribonucleic acid (RNA), obtained from any synthetic or biological source. These include plasmid DNA's, genomic DNA, synthetic DNA's or RNA's, DNA's synthesized by polymerase chain reaction (PCR), polynucleotides, oligonucleotides, synthetic phosphodiester oligonucleotides, methylphosphonate oligonucleotides, phosphorothioated oligonucleotides, morpholino nucleic acids, morpholino oligonucleotides, modified nucleic acids, nucleic acid mimetics, chimeric nucleic acids, nucleoside polymers, nucleotide polymers, peptide nucleic acids (PNA), and PNA-DNA chimeras. Nucleic acids can be single stranded but also include duplex, triplex (single strand complexed with double stranded), and polyplex forms. Also suitable are any nucleic acid polymers comprising the genetic bases adenine (A), cytosine (C), guanine (G), thymine (T), or uracil (U) wherein the polymer backbone is selected from, but is not limited to, a ribose, deoxyribose, ribose-phosphate, sugar-phosphate, peptide, morpholine, morpholine-phosphate, and ribose analogues.

Antisense Oligonucleotides

Single stranded oligodeoxynucleotides, typically less than 40 base pairs (bp) in length, that are Watson-Crick hybridizable to a specific mRNA that codes for a target protein. After the anti-sense strand binds to the mRNA, the message is cleaved by RNAse H, and synthesis of the specific protein is blocked. Anti-sense oligonucleotides are highly sequence selective, and must hybridize to a region of mRNA that elicits an effective RNAse H response.

SiRNA

Double stranded RNA's comprising an antisense and sense strand, that have sequence specific binding to a mRNA target. The siRNA can form an RNA induced silencing complex (RISC) which can bind to the mRNA target in a cell and cause cleavage of the message by dicer enzyme.

Antisense Therapy

Defined as therapeutic methods that operate primarily by anti-sense mechanisms. In anti-sense therapeutics it is desired to cause hybridization of the anti-sense oligonucleotide to the target mRNA or DNA in the cell, thereby inducing cleavage of the message and silencing of the target protein. Without carriers, ODN's and siRNA's are shown to be well tolerated and have negligible biological effects on cells. A significant limitation of anti-sense therapies against cancer is the lack of uptake into the cell, but also lack of potency of the mechanism itself.

Masking Agents

Preferred masking agents are substantially bio-compatible and are optionally incorporated in the present invention to provide increased molecular weight, stability against nuclease attack, prolonged circulation, and other favorable pharmacokinetic properties. Suitable masking agents include lipids, vitamins, cholesterol, polyethylene glycol, polyalkylene glycol, polyols, sorbitol, PEG grafted polyols, polyglutamate, hydroxypropylmethacrylamide (HPMA), HPMA copolymers, peptides, fatty acids, alkyl chain, polysaccharides, human serum protein, avidin, cyclodextrin, dextrans, and PEG derivatives (i.e. PEG-cholesterol, PEG-lipid, PEG-corn oil, and pharmaceutical grade PEG-surfactants).

Transfection Agent

Transfection agents are defined as agents that facilitate the intracellular transport of nucleic acid. Cationic transfection agents are positively charged monomers or polymers, that are optionally combined with a nucleic acid to facilitate transduction across cellular membranes. Many transfection agents are disclosed in the art such as polyethylenimine (PEI), polyalkylenimines, polyarginines, polyamines, protamines, polylysines, fusogenic peptides, polyamidoamine dendrimers (PAMAM, Dendritech Inc.), pegalated cationic polymers, cationic polymer conjugates (i.e. PEI-cholesterol, polylysine cholesterol, etc.), chitosans, cationic dextrans, cationic cyclodextrins, and cationic lipids such as dioleoyltrimethyl-ammonium propane (DOTAP), N-[1(-2,3-dioleoyloxy)propyl]-N,N,N-trimethylammonium chloride (DOTMA), N,N-dioleyl-N,N-dimethylammonium chloride (DODAC), Lipofectamine™, and Gene Porter™.

Drug Conjugate

“Drug conjugate” are any modified forms of a drug, and more preferably, any suitably active forms, wherein the drug is grafted to another moiety.

Pro-drug

“Pro drug” is a drug or drug conjugate that must be activated by a chemical process, i.e. by either forming or breaking a chemical bond.

Pharmaceutical

Pharmaceuticals are any substances, and more preferably any bio-active substances, or formulations thereof, that can be suitably administered to a patient for the purpose of treatment, prevention, or diagnosis of a disease, condition, or malady. The composition more preferentially relates to bio-active drugs and compositions thereof, that are useful to cause a measurable biological response in a cell or organism, and still more preferentially to bio-actives and compositions that are useful in the treatment or prevention of a disease, condition, or malady. Pharmaceuticals include any bio-active agents that are administered to the patient by oral, parenteral, intravenous, transmucosal, pulmonary, depot, or other means. The term “pharmaceutical” also applies to any bio-actives or therapeutic compositions that are suitable for pharmaceutical research and development.

The Composition

This invention relates primarily to compositions of novel, nucleic acid-drug compositions wherein a drug is non-covalently bound to a nucleic acid, herein referred to as the “nucleic acid carrier”. More specifically, the invention relates to the use of nucleic acids, but also nucleic acid fragments, nucleotides, nucleosides, and polymers thereof, to bind with drugs, reversibly inactivate drugs, co-transport drugs, and provide nucleic acid targeting of drugs.

When a suitable nucleic acid carrier and drug are combined in solution, they self-assemble to form a nucleic acid-drug particle, or complex, capable of delivering a drug to a sub-cellular location. The complexing of the nucleic acid carrier and drug is desired so that it can reversibly inactivate or mask the drug's activity during co-transport to a target. The invention can be used in the formulation of many kinds of therapeutic entities, such as organic drugs, drug conjugates, and protein drugs, provided that the nucleic acid carrier and drug have sufficient mutual affinity. In other aspects, the invention can be used in the nucleic acid targeting of many other types of drugs that normally do not associate with nucleic acid by conjugating the desired drug to a nucleic acid-binding spacer (NABS). With optimized nucleic acid drug formulations, it is desirable to form compositions that are substantially “drug-free”, such that the concentration of unbound or free drug is minimized: By utilizing nucleic acid carriers and drug combinations that have sufficient mutual affinity, the nucleic acid can mask the drug and toxicity or activity can be minimized prior to delivery to the target site. In addition to drug masking, and other benefits, it is shown herein that nucleic acid drug complex can target therapeutic agents to specific tissues based on cellular factors that relate to uptake. Nucleic acid targeting can increase accumulation of drugs in solid tumors while at the same time reducing accumulation in off-target sites. In other embodiments, nucleic acid targeting can be controlled to enhance sub-organ accumulation of therapeutic agents based on the hydrophobicity, backbone chemistry, cell surface receptors, and other factors. In the effective targeting of nucleic acid-drug carriers, it is therefore an objective of the invention to utilize nucleic acid-drug compositions which effectively bind the drug of interest, so that an association between nucleic acid and drug exists until the nucleic acid-drug carrier reaches the target, and effective targeting by the nucleic acid-drug carrier can be realized. Finally, the novel properties of the invention are shown to be influenced in surprising ways, by selection of drug loading, by the type of drug, and by the structure of the nucleic acid.

In the simplest definition, the nucleic acid-drug carrier comprises a drug and a nucleic acid carrier with one or more drug binding sites, such that it contains nucleic acid residues capable of contacting with the drug. Binding of the nucleic acid to the drug is preferably non-covalent, such that the drug can be released from the carrier. The nucleic acid-drug carrier can be represented by the formula NAC-X where NAC is the nucleic acid carrier and X is the drug. When NAC and X are combined to form the composition, the concentration of free X is reduced in solution such that NAC is at a sufficient concentration to sufficiently bind X. It is shown that NAC-X can be purified from X by the use of column chromatographic methods, in order to maximize the concentration of NAC-X in solution. However, it is understood that an equilibrium exists between NAC-X and free NAC and X at all times, based on the molar binding constant. It is preferred that NAC and X are combined at a molar ratio which minimizes free X

This invention is primarily advantageous in the formulation of drugs which have an affinity for nucleic acid, due to their ability to fit within the nucleic acid helix or along the minor or major groove of DNA. Without limiting the invention, drug binding to the nucleic acid is complex and is believed to occur by a number of different processes, such as intercalation or groove binding, but also charge interactions. In double stranded nucleic acid, the planar, heterocyclic drugs can fit into the space between the nucleic acid strands. Intercalator drugs such as anthracene derivatives, measure approximately seven angstroms across which allows these drugs to fit into the roughly ten angstrom space between the nucleic acid strands. The precise fitting of these molecules within the nucleic acid structure creates close contact and electronic π-π interaction between the aromatic groups of the drug and the aromatic groups of the nucleic acid bases. In addition, the nucleic acid binding site includes regions which may interact with nitrogens or other electronegative atoms in the drug molecule, which contribute by hydrogen bonding and potentially by interaction with negatively charged phosphates. The nucleic acid may also bind to a drug at the major or minor groove. Heterocyclic drugs which are too large to fit between strands may bind in the minor or major groove of the nucleic acid by conforming to the shape of the helix.

DNA Intercalating Drugs

DNA intercalating drugs are a class of biologically active molecules which function primarily in their capacity to intercalate DNA. DNA intercalating drugs that are considered clinically safe and are suitable for administration in human or veterinary medicine are highly preferred.

Without limiting the invention, the following are provided only as a list of potential drug candidates which are contemplated for use in the present invention and are by no means exhaustive. Suitable DNA intercalator drugs include, doxorubicin (DOX), mitoxantrone (MTN), actinomycin, daunomicin, daunorubicin, idamycin, cerubidine, bleomycin, actinomycin D, echinomycin, nitracrine, anthracycline antibiotics, anthraquinone antibiotics, anthracenediones, mithramycin, porphyrins, porphyrin derivatives, benzothiopyranoindazoles, acridines, pyrazole acridine, acridine derivatives, quinoxalanes, quinoxalane derivatives, and ecteinascidin 743.

Groove Binding Drugs

Drugs can also cause cytotoxic effects based on their binding in the minor groove of DNA. Suitable minor groove binding drugs which have utility in the present invention include berenil, netropsin, and distamycin.

Nucleoside Drugs

Also suitable are nucleoside drugs, nucleotide drugs, conjugates, and derivatives that bind DNA and are cytotoxic by inhibition of the binding and function of a protein critical for the function of viral or cellular DNA. Antineoplastic agents such as cytarabine are synthetic nucleosides and are cell-cycle specific cytotoxic agents. Cytarabine effects cells primarily in the S phase. Other nucleoside based antineoplastic agents are known, such as 2′-deoxy-2′,2′-difluorocytidine (gemcitabine). Also included are anti-viral nucleoside drugs, 3′-azido-3′-deoxythymidine (AZT), didanosine, zalcitabine, lamivudine, 1592u89, stavudine, and RT inhibitor analogues, such as Epivar™, Hivid™, Zerit™, and Videx™, Ribovirin™, and Viramidine™. Also included are any phosphorylated forms, such as mono, di, or tri phosphates of nucleoside drugs and nucleoside analogues.

Anti-Mitotic Agents

Molecules which interfere with mitosis in the cell are referred to herein as anti-mitotic drugs. Anti-mitotic agents such as colchicine, colcemid, and nocadazole inhibit addition of tubulin to microtubules, causing depolymerization of the microtubules. Vinblastine and vincristine cause tubulin molecules to crystallize and aggregate. Taxanes, such as paclitaxel and docetaxel, bind to tubulin and inhibit mitosis by causing formation of microtubules from free cellular tubulin or stabilization of microtubules due to polymerization. Tubulin dimer is a polar, 50K globular protein, and is highly present in brain and nerve cells (10-20% of total protein), particularly in the axons and dendrites [B. Alberts, D. Bray, J. Lewis, M. Raff, K. Roberts, J. D. Watson, Mol. Bio. Of The Cell, 2:650-659 (1989)]. Paclitaxel is also shown to bind to the anti-apoptotic protein BCl-2 and to inhibit the growth of prostate cancer cells [Haldar, S., Chintapalli, J. Croce, C. M., Paclitaxel induces Bcl-2 phosphorylation and death of prostate cancer cells, Cancer Res. 56:1253-1255 (1996)]. Paclitaxel is known to bind in the major groove of DNA, and is selective primarily toward A/T sequences [G. Bischoff et al. DNA binding of drugs used in medicinal therapies, Curr. Med. Chem. (2002) 9:321-348]. It is shown in the exemplary disclosure that aqueous formulation of paclitaxel with a double stranded polynucleotide can significantly enhance the solubility of paclitaxel.

Drug-NABS Conjugates

The nucleic acid targeting of many kinds of drug entities such as anti-mitotic drugs, intercalator drugs, groove binding drugs, steroids, alkaloids, anti-neoplastics, peptide drugs, DNA alkylators, platinates, nucleoside drugs, nucleotide drugs, among others, can be enhanced by conjugation of the drug with a nucleic acid binding spacer (NABS). The nucleic acid binding spacer is optionally selected from a compound that intercalates with DNA or binds in the minor or major groove of DNA. The NABS can also comprise a quaternary nitrogen to enhance affinity of the drug with the negatively charged phosphates. Many drug conjugates are available or have been disclosed that are potentially useful as drugs in the present invention. For example, DNA alkylating drugs that are conjugated to nucleic acid binding moieties, such as platinum-acrindinylthiourea disclosed by H. Baruah et.al. and imidazotetrazines conjugated to DNA binding peptides (SPKK)2-NH2, disclosed by J. Arrowsmith et al. Cancer Research Laboratories, UK. The NABS can be conjugated to the drug by a variety of means known in the art of drug conjugation, to provide stable association between the NAC and the drug conjugate. The anti-mitotic drug, paclitaxel, can be conjugated to the NABS using similar methods known and described in synthesis of paclitaxel pro-drugs and paclitaxel derivatives such as Flutax [Diaz et. al. J. Biol. Chem. 275:26265-26276 (2002) and Papot et.al. Curr.Med.Chem 2:155-185 (2002)]. Nucleoside drugs can be conjugated to the NABS via ester, amide, hydrazone or other suitable linkages known in the art. Preparation of AZT-3,6 diaminoacridine conjugate and complexation with HSDNA carrier are disclosed herein. Similarly, many anti-neoplastic drugs such as 5-fluorouracil, can be conjugated to a DNA intercalator via an amide bond or other suitable bond. The linkage or bond in the NABS-drug conjugate can optionally be chosen to provide enzymatic activation of the drug once inside the cell [Madec-Lougerstay et.al. J. Chem. Soc. Perkin 1369 (1999) and Papot et.al. Curr.Med.Chem 2:155-185 (2002)]. It is preferred that the drug-NABS conjugate can sufficiently associate with the nucleic acid carrier by non-covalent means, so that the drug conjugate can be co-transported with the nucleic acid to the desired address.

Polymeric Drug Conjugates

Recently, pegalation has been utilized to produce PEG-drug conjugates, such as PEG-doxorubicin, and PEG-protein based drugs such as PEG-a-interferon. Other polymers such as HPMA have also been described in the synthesis of conjugated drugs such as HPMA-doxorubicin, among others. Polymeric drug conjugates are useful as drugs in the present invention provided that the drug can associate sufficiently with the NAC to co-transport with the NAC to the active site.

Other Therapeutic Agents

Other examples of useful therapeutic agents include folate antagonists (methotrexate), trichostatin A, dipyridamole, cyclosporine, daunorubicin, dactinomycin, idarubicin, pentostatin, cladribine, fludarabine, hydroxyurea, asparaginase, topotecan, troglitazone, dacarbazine, fludarabine, carmustine, chiroambucil, prednisolone, DHEA, camptothecin, etoposide, bleomycin, phleomycin, aminopterin, daunorubicin, 5-bromo-2′-deoxyuridine, cytosine β-D-arabinofuranoside, duocarmicins, vinca alkyloids, plant derived toxins (ricin, lectins), enediynes (chalicheamicins, maytansines) and CC-1065. Also included are non-nucleoside RT inhibitors i.e. nevirapine, loviridine, DMP266, delavirdine, and protease inhibitors; nelfinavir, 141w94, indinavir, saquinavir, and ritonavir. DNA binding agents; carmustine, melphalan, carboplatin, and diammineplatinium(II) HCl. Also included are natural or synthetic toxins and cytotoxins; diphtheria toxin, Tf-CRm107 [anti-cancer DT mutant, Hagihara et al Canc. res. 60:230-234 (2000)], pertussis toxin, digitoxin, aflatoxin B, peptide toxins (grammatoxin), cholera toxin, neurotoxins, botox, opioids, opiod peptides, neuropeptides, protamine sulfate, benzocaine, morphine, fentanyl, ziprasidone mesylate, melatonin, anti-depressants, anti-convulsants, etc. microtubule inhibitors; auristatins, dolastatin 15, demecolcine, nocodazole, myoseverin, kinase inhibitors, anti-fungals, voriconazole, and pirfenidone, and antibiotics; neomycin, gancyclovir, etoposide, bafilomycin, actinomycin, amoxicillin, amphotericin, ampicillin, antimycin, aphidicolin, fumagillin, coumermycin, concanamycin, brefeldin A, mithramycin, hygromycin, praziquantel, puromycin, tetracycline, streptomycin, tubercidin, polymyxin, ofloxacin, and vancomycin.

The Nucleic Acid

The nucleic acid can be a DNA or RNA obtained from any synthetic or natural source. Suitable DNA can be isolated from the cells of organisms, preferably from sources that are non-immunogenic, and suitably purified or restriction enzyme digested to obtain desired uniformity and molecular weight. More preferably, nucleic acids are synthesized with solid state phosphoramidate chemistry, using automated methods known in the art, purified by HPLC, and sequence verified by mass spectrometry. Suitable nucleic acids can also be obtained in large quantity by use of the polymerase chain reaction. Suitable plasmid DNA's can be isolated from bacteria using conventional plasmid purification techniques which minimize contaminating RNA's and endotoxins. Genomic DNA can be isolated from cells using conventional DNA isolation techniques, which typically require sodium hydroxide or enzymatic lysis followed by phenol-chloroform extraction, ethanol precipitation, or affinity chromatography to isolate the DNA. Nucleosides and nucleotides that are suitable in the invention are readily obtained from commercial sources. Customized synthetic nucleic acids that are suitable in the present invention are also available commercially from suppliers such as GeneTools Inc. and Invitrogen Inc. San Diego, Calif.

In the preparation of nucleic acid-drug pharmaceuticals, nucleic acids derived from synthetic sources are highly advantageous in the invention because they have a well defined structure and concise molecular weight. By using automated nucleic acid synthesis, and HPLC purification, the molecular weight and sequence of the nucleic acid carrier can be carefully controlled. In addition, synthetically derived nucleic acids are substantially free of biologically derived contaminants such as endotoxins, which are potentially co-purified from polynucleotides obtained from biological sources.

The nucleic acid structure can vary somewhat, depending on the desired uptake profile, bio-distribution, drug loading, and other factors. In-vitro studies of the carriers show that the molecular weight of the nucleic acid carrier influences performance, likely because of its influence on uptake into the cell. The molecular weight of the nucleic acid will likely influence uptake but also distribution and circulation time of the nucleic acid carrier. The double stranded carriers are shown to be highly useful in the present invention and are more preferred carriers of certain drugs, likely due to stronger binding as well as higher stability against nuclease attack.

In nucleic acid-drug carriers, modifications to the nucleic acid backbone can be beneficial to provide useful pharmacokinetic properties. The chemistry and biological properties of modified DNA's are an intensely researched field, and many are known in the art which can provide utility in the present invention. For example, backbone modifications can enhance the stability of the carriers against nuclease degradation, reduce accumulation and passage via the kidneys, increase distribution in tissues, and reduce plasma clearance. One potentially useful modification to the nucleic acid is the replacement of non-bridging oxygens with sulfur to form a phosphorothioated nucleic acid. The circulatory half-life of phosphorothioated oligonucleotides is shown to be significantly greater than diester oligonucleotides, which is likely due to the higher affinity of phosphorothioates for plasma proteins. For example, studies in mice show that single stranded, phosphorothioated ODN's, administered by a single intravenous bolus, exhibit a half life of 11 hours and a steady-state terminal elimination half-life of 22 hours (Raynaud et al 1997). In contrast, in-vivo studies of unmodified DNA's have shown rapid clearance from plasma (Crooke et al 2000, Sands et al 1994). Nucleic acid-drug carriers composed of phosphorothioated nucleic acid are advantageous because of their association with plasma proteins and increased distribution into target areas such as tumors, due to increased circulation time among other factors.

In the body, the distribution of NAC-drug is expected to be largely dependent on the properties of the nucleic acid. However, the intercalation of certain drugs and the resulting effect of drug binding on the structure of the nucleic acid might also influence in-vivo performance in unexpected ways.

Nucleic acids are biodegradable and well tolerated, and the neutrality of the nucleic acid carrier molecule is an advantage in the significant drug masking effects of the invention. In nucleic acid-drug carriers, the pharmacological effects on cells are therefore primarily dependent on the function of the drug. Clinical studies show that nucleic acids are well tolerated in the adult. Maximum tolerated doses of phosphorothioated ODN's in humans have been in the range of 147 mg/m2 or plasma level of about 4 ug/ml (Waters 2000b). Maximum tolerated doses of phosphodiester oligonucleotides can be higher since these do not bind serum proteins and are less likely to cause activation of the compliment system or the immune system.

Uncharged nucleic acids such as peptide nucleic acids, methylphosphonate oligonucleotides, and morpholino oligonucleotides are known in the art and are available from commercial sources such as BioSynthesis Inc. and GeneTools Inc. Peptide nucleic acids (PNA) generally comprise a 2-aminoethyl-glycine backbone linked to nucleobases A, C, G, T via a carbonyl linker and are synthesized by standard solid state methods used for peptides [Buchardt et al, Neilsen et al., and Ray et al., J. FASEB 14:1041-1060(2000)]. PNA's can optionally incorporate positively charged lysines (in place of glycine) or negatively charged groups to enhance solubility. Morpholino nucleic acids comprise subunits of morpholine rings linked to adenine, cytosine, thymine, or guanine. The morpholino subunits are then conjugated by non-ionic phosphorodiamidate linkages to form an oligonucleotide chain. Uncharged nucleic acids have been shown to have low biological effects on cells, as compared with phosphodiester and phosphorothioates. The reported lack of intracellular activity of these nucleic acids is thought due to very low intracellular uptake based on the high polarity of the nucleotides [Stein C. A. Lebedeva I., Antisense Oligonucleotides: Promise and Reality, Annu. Rev. Pharm Tox. 41:403419 (2001)]. However, uncharged oligonucleotides can have significant advantages for enhancing the preferential uptake of the nucleic acid-drug carriers by reducing their uptake into non-target cells or tissues and in reducing their accumulation in off-target sites.

Aptamer

Nucleic acid ligands or “aptamers”, are nucleic acids capable of binding to other molecules in a sequence specific manner. Aptamers are typically screened and selected against a target from large synthetic DNA or RNA libraries and can discriminate based on folding or sequence for a particular molecule, such as a protein, a cellular antigen, or a small molecule. Naturally occurring aptamers are also known. The binding of aptamers is some what different from intercalation or groove binding and typically involves folding of the nucleic acid around the molecule. By selection of appropriate length and sequence, nucleic acid ligands or aptamers can be synthesized against a broad array of molecular targets. To date, approximately one hundred aptamers have been identified against compounds such as moenomycin [Schurer et al. Bioorg Med. Chem. 2001], tobramycin [Patel et al, Nat. Struct. Bio. (1998)], vitamin B12, cocaine [Stojanovic et al, J. Am. Chem. Soc (2001)], and fluorescein, among others. Aptamer-drug complex can be useful in the present composition to facilitate novel drug co-transport, masking, function, and nucleic acid targeting. Moreover, NAC's comprising an aptamer structure or sequence can be highly useful for binding drugs that substantially lack affinity for DNA, e.g. drugs that substantially do not intercalate or bind nucleic acid in the minor or major groove, to provide effective nucleic acid targeting of the drug. It is preferable that the NAC-drug is combined at a mass ratio that is sufficient to substantially bind the drug, preferably such that the percentage bound drug in the formulation ranges from between 0.1% and 100% and still more preferably at between 1% and 100%.

Like their anti-sense oligodeoxynucleotide (ODN) or siRNA counterparts, aptamers have been proposed to bind an endogenous protein or DNA target in the body. For example, aptamer drugs, such as anti-VEGF aptamer, pegaptanib sodium (Mucagen™), have been proposed to inhibit the function of an endogenous protein to reduce the growth of blood vessels. However, like anti-sense ODN's and siRNA's, the potency of aptamers as drugs have been limited by problems of low biological activity and poor cellular uptake.

Anti-toxins

Nucleic acids and nucleic acid carriers can be utilized to reduce the toxicity of a drug in the body by administering a suitable nucleic acid carrier that binds the drug and facilitates its rapid removal from the circulation. Currently there are no suitable means for treating overdoses from anthracycline anti-biotics. Nucleic acid carriers are bio-compatible, non-immunogenic, and can be administered at sufficient concentration for extended periods as necessary to achieve a therapeutic benefit. NAC's can be utilized to treat overdoses of other drugs, and drugs of abuse, such as cocaine, heroin, etc by administering an appropriate nucleic acid carrier that binds the drug, to reduce toxicity and enhance clearance. NAC's and more particularly, NAC aptamers, can also provide a suitable means to remove other harmful toxins, or toxic substances that may be present in the body such as toxins produced from bacteria, pesticides, venoms, infection, among others.

NAC's Composed Of Nucleosides

It was discovered unexpectedly that nucleosides and nucleotides associate strongly with DNA intercalating drugs and minor groove binding drugs. NAC's comprising nucleosides such as adenosine, cytadine, guanosine, and thymidine were found to effectively bind mitoxantrone and doxorubicin and significantly reduced drug toxicity in cell culture. Nucleosides are typically composed of a pentose, 5 carbon sugar ring grafted with a nucleobase adenine, cytosine, guanine, thymine, uracil and include deoxy, dideoxy, azido, thio, phosphorylated (nucleotide), and other derivatized forms. The binding of the nucleoside with the drug can mask drug toxicity, enhance solubility, and target the drug to a desired address. Phosphorylated nucleosides are highly preferred since these are found to bind more strongly to DNA binding drugs. Also useful are any polymers, conjugates, derivatives, and analogues of nucleosides, such as coenzyme A (CoA), dinucleosides, nucleoside-fatty acid conjugates [(Seki et al, Pharm. Res. 7:948 (1990)], and nucleoside-cholesterol [Pannecoucke et al., Tetrahedron 50:1173(1994)], that are highly suitable to provide drug-nucleoside association and co-transport across cell membranes. Also useful are any nucleosides conjugated to masking agents.

In a dosage form suitable for parenteral administration, the nucleic acid can be combined with the drug in an aqueous formulation of saline, dextrose or other suitable aqueous vehicle suitable for administration. It is preferred that the drug and the nucleic acid are combined in water or other suitable medium, prior to dilution in saline or media, in order to prevent inhibition of drug binding to the nucleic acid. The dosage form can be sterilized by a variety of means, and can optionally take the form of a concentrate that can be diluted in saline or other suitable vehicle prior to injection. It is preferred to minimize the amount of free drug in the injectable solution, such that the mass ratio of nucleic acid to drug is sufficient to bind most of the drug, and to limit free drug in the bloodstream. The optimal mass ratio for minimizing free drug has been found to vary depending on the drug binding characteristics. In preferred embodiments, the concentration of nucleic acid in the dosage form should be selected which provides sufficient drug loading characteristics. For example, in drug loading studies of nucleic acid-mitoxantrone (NAC-MTN) it was found that double stranded DNA's provide minimal free or unbound drug in aqueous solutions at about 50% weight to weight (w/w) of MTN to nucleic acid carrier, such that the free drug is reduced to about 3% or less. However, of nucleic acid-doxorubicin (NAC-DOX) it was found that free DOX was minimized at up to about 10% w/w of DOX to nucleic acid carrier. The percentage loading possible for other nucleic acid binding drugs will vary depending on their binding characteristics, and can be determined by column chromatography, spectrophotometry, and cytotoxicity assay methods as described herein. It preferred embodiments, the w/w percentage of drug that is bound to the nucleic acid in the composition optimally ranges between 1% and 100%, but more preferably between 5% and 100%, and still more preferably between 40% and 100%.

Other DNA binding drugs, such as cisplatin, are described as binding DNA via hydrogen bonding, by charge-charge interaction with the phosphates, and by forming crosslinked adducts between glutamine bases. One objective of formulating cisplatin with a nucleic acid carrier is in preserving the activity of cisplatin prior to administration. Because cisplatin is base selective, the sequence and structure of the nucleic acid can be important in modulating the performance of NAC-cisplatin. It is an objective of the invention to bind cisplatin to the NAC such that cisplatin activity is minimized during transport but activity can be restored after it reaches a target. For example, by selection of an appropriate nucleic acid sequence, such as a nucleic acid sequence that is substantially free of guanine bases, nucleic acid carriers can be designed to bind with cisplatin but resist the formation of covalent bonds. The nucleic acid-cisplatin carrier would selectively enter cells which showed preferential uptake, and cisplatin could then actively form Pt adducts with cellular DNA. Also suitable are dosage formulations comprising a single stranded DNA that can sufficiently bind cisplatin but inhibit crosslinking between strands. Optionally, compounds containing thiols that are known to inhibit the binding of cisplatin can be formulated in a dosage form of the nucleic acid-cisplatin to stabilize the composition during storage [Sadowitz P. D. et al, Kinetics Of Cisplatin Binding To Cellular DNA and Modulations By Thiol Blocking Agents and Thiol Drugs, Drug Met. and Dis. 30:183-190 (2002)]. Nucleic acid targeting of cisplatin can also be achieved by forming a conjugate of cisplatin further comprising a nucleic acid binding moiety or nucleic acid binding spacer (NABS), that can provide sufficient binding and association of cisplatin with the carrier but without chemically altering cisplatin activity.

The nucleic acid carrier is preferably selected that binds the drug sufficiently to prevent pre-mature dissociation of the nucleic acid-drug complex due in part to competitive binding of the drug with other components in the blood stream such as serum proteins, lipids, and red blood cells. Dissociation of the drug from the nucleic acid-drug complex and subsequent complexing and association with proteins or lipids can circumvent the targeting and masking of the nucleic acid and potentially cause rapid absorption of these drugs into cells, leading to toxicity. The drug loading percentage, on weight:weight (w/w) basis can be adjusted to limit free drug which is available for extraneous binding. For the purposes of complete encapsulation of some types of drugs, it has been discovered that double stranded nucleic acid is advantageous to single stranded DNA.

In designing and formulating a nucleic acid carrier and a particular drug, the drug loading, targeting, uptake, release and other parameters are worked out by experiment. Novel methods are described herein for the preparation of nucleic acid carriers with doxorubicin and mitoxantrone that are applicable to the identification, screening, and production of other nucleic acid drug carrier compositions. In the physical determination of drug loading limits, and drug release capacity, the nucleic acids and drugs are combined at different mass ratios and assayed for secondary drug peaks by column chromatography. Cell culture assay are also useful in determining drug loading limits, and are in some cases more accurate in determining actual drug masking. For example, in cell culture toxicity assay, cells are incubated with varying concentration of nucleic acid-drug complex at a fixed drug loading percentage. If toxicity is not observed to change across a dilution curve, the free drug in the formulation is negligible. Efficacy of drug release, activity, and carrier performance is also assayed by cytotoxicity, as described herein. Finally, nucleic acid targeting of the drug carrier can be screened by comparing uptake in different cell lines and by measuring uptake under varying cell-plating and growth conditions. The methods described herein can be used to screen and formulate nucleic acid-drug compositions that are substantially free of unbound drug, efficacious against a target, and show nucleic acid targeting.

Paclitaxel was first isolated from the bark of taxus brevifolia. Paclitaxel is approved for the treatment of metastatic ovarian cancer and breast cancer, among others. Paclitaxel is shown to arrest cellular mitosis in the G/M phases by inducing the assembly of tubulin dimers into microtubules and by stabilizing microtubules against disassembly in the cell. Paclitaxel is also believed to induce apoptosis by phosphorylation of the anti-apoptotic Bcl-2 protein, a protein that is over expressed in many types of cancers. Because paclitaxel is extremely hydrophobic, it is currently formulated for injection with ethanol and pegalated castor oil, which can cause hypersensitive immune responses and requires pre-medication. Paclitaxel chemotherapy is effective but is associated with significant side-effects due to uptake into numerous non-target cells of the body. The significant cytotoxic action of paclitaxel limits the dose that can be applied, reduces the effective concentration in the tumor, and increases the risk of acquired tumor resistance. Hence, a significant impediment to current paclitaxel based therapies is the acquisition of tumor drug resistance. Drug resistance is believed to occur by over expression of P-glycoproteins in the cell membrane that actively pump paclitaxel and other small drugs out of the cytoplasm. Nucleic acid-paclitaxel carriers can reduce tumor cell resistance by providing a unique pathway of uptake and intracellular trafficking of paclitaxel, and circumventing P-glycoprotein mediated resistance. In addition, nucleic acid-paclitaxel carriers can significantly enhance the therapeutic index of paclitaxel by providing nucleic acid targeting to paclitaxel, thereby increasing paclitaxel concentrations in tumors, while reducing paclitaxel in non-target cells, and reducing the likelihood of acquired resistance. Furthermore, nucleic acid carriers can enhance the solubility of paclitaxel, and provide a bio-compatible and non-immunogenic alternative to current paclitaxel formulations of ethanol/chremaphor. It is shown in the disclosure that formulation of paclitaxel with a nucleic acid can enhance the solubility of paclitaxel by as much as 200% compared with water alone. Greater solubility of paclitaxel can be achieved by utilizing a nucleic acid carrier that has higher affinity for paclitaxel. Paclitaxel is currently difficult to formulate with the HSDNA carrier, likely because the binding and association of paclitaxel-HSDNA is not very strong. It is possible that paclitaxel more strongly associates with serum proteins in the media, than with the genomic HSDNA (100-3K bp), and so the nucleic acid binding are diminished. The case illustrates the importance of selection of appropriate nucleic acid sequence and structure in the formulation of nucleic acid-drug carriers. In compositions of nucleic acid-paclitaxel carriers, the nucleic acid is preferably selected that has a sequence affinity for paclitaxel or contains high ratio of nucleotide bases that are known to bind paclitaxel, such as thymine and adenine. For example, nucleic acid sequence libraries that are selective for paclitaxel can be screened by high throughput methods known in the art of preparing and selecting aptamers. Since aptamer-antigen binding constants can be on the order of antibody-antigen, potential problems can occur if the aptamer does not sufficiently release the drug once the nucleic acid-paclitaxel complex enters the cytoplasm. More preferred nucleic acid-paclitaxel compositions can be identified from aptamer libraries by tissue culture screening methods, disclosed herein. In cell culture screening assay, paclitaxel is combined with the nucleic acid at varying weight to weight ratio, and the complexes are incubated for several days with cells, such as BHK21. The toxicity of the resulting nucleic acid-paclitaxel complexes is determined by MTT cell viability assay.

Nucleic acids containing a peptide sequence that binds paclitaxel, such as nucleic acid-peptide conjugates, can be highly useful in the present invention for providing drug masking, solubility, and nucleic acid targeting of paclitaxel. Bacteriophage screening has shown that the amino acid sequence required for the binding of paclitaxel is likely in the range of between 10 and 20 amino acids and can be as few as 5-7 [Rodi D. J., Makowski L., Similarity between the sequences of paclitaxel selected peptides and the disordered loop of the anti-apoptotic protein, Bcl-2, (1998)]. Paclitaxel binding sequences have been identified in BCl-2 protein and β-tubulin [Nogales E., S. G. Wolf, K. H. Downing, Structure of the αβ tubulin dimer by electron crystallography. Nature 391:199-203 (1998)]. Similarly to non-peptidic nucleic acid carriers, the suitability of peptide nucleic acids in the formulation of paclitaxel can be determined utilizing cell culture toxicity assay. Additional cell uptake assays, as described herein, can also be used to screen the suitability of peptide nucleic acid in providing drug masking and cell selective targeting of paclitaxel.

Chimeric peptide-nucleic acids comprising a nucleic acid and a peptide sequence that is complimentary to the nucleic acid, can be advantageous in the formulation of paclitaxel. Protein-nucleic acid binding can be very strong, as in the case of aptamer-enzyme binding. A principal objective of the invention is to reduce the availabilty of unbound paclitaxel in the bloodstream while at the same time enabling the efficacious release and function of paclitaxel once it enters the target cell. Nucleic acid carriers that have substantial affinity for paclitaxel can provide greater drug masking and nucleic acid targeting of paclitaxel, reducing the rate of uptake of paclitaxel into off-target cells and increasing accumulation in cells and tissues that show preferential uptake, such as rapidly dividing cancer cells.

Increasing the resistance of the nucleic acid carrier to nuclease degradation can be important for preventing premature release of the active drug and for consistent performance of the carrier. The intercalation or binding of drugs to the nucleic acid may enhance the stability of the carrier against nucleases, since intercalators are known to disrupt the structure of DNA by expanding and unwinding the duplex. The use of double stranded nucleic acid carriers can also significantly enhance in-vivo stability. Methods for stabilizing nucleic acids against enzymatic degradation are known in the art, such as replacing non-bridging oxygens with sulfur to produce phosphorothioated nucleic acid. Modification to the 2′ end of the nucleic acid with a NH2 are also known in the art to induce enzymatic resistance.

Doxorubicin is an anthraquinone antibiotic isolated from streptomyces peucetius. It functions by intercalation of DNA, interference with DNA polymerases, generation of free radicals, and is cell phase non-specific. Doxorubicin, also known as adriamycin, is an antineoplastic drug used in the treatment of cancers which are refractory for first line therapies. Formulation of doxorubicin in a nucleic acid carrier produced surprising results. In drug binding studies utilizing column chromatography, the nucleic acid-DOX carrier (NAC-DOX) showed a primary peak at relatively high loading of DOX, about 10% by weight:weight of DOX to the nucleic acid and free drug was not detected. These data, combined with absorbance and fluorescence from binding studies of the nucleic acid-DOX complex, show that the carrier is surprisingly effective at loading doxorubicin.

The NAC-DOX complex was tested for stability in hank's buffered saline (HBSS) at pH 7. Doxorubicin solutions were stable at reduced pH but became unstable at pH 7 and were prone to precipitation in buffered saline and in media. Surprisingly, the NAC-DOX was stabilized against precipitation even after prolonged storage.

In-vitro studies of the toxicity of NAC-DOX were performed in MCF-7 breast cancer cells. It was found that when DOX was formulated in the nucleic acid carrier, the toxicity of doxorubicin was substantially reduced (25 fold) in MCF-7 cells that where not actively dividing. However, in non-confluent and freshly plated MCF-7's, toxicity of the nucleic acid-DOX carrier was significantly greater.

To explore the nature of the NAC-DOX binding, other in-vitro studies (EX050404) were designed to test for the presence of free drug at different drug loading levels. While keeping the loading fixed at either 2%, 4% ,or 8%, the total concentration of NAC-DOX was increased in confluent, MCF-7 cells and toxicity was monitored in the cells over time. It was discovered that at 8% loading, toxicity did not increase as a function of total NAC-DOX and show that free drug is not present or is minimally present at levels which could not be detected.

These data were surprising and unexpected given the fact that no doxorubicin toxicity was observed, even when the effective doxorubicin concentration in solution was far in excess, about 25 fold greater than the amount needed to cause obliteration of 100% of the cells.

Pharmacological studies in humans with doxorubicin show that doxorubicin is slowly cleared with only 4-5% of administered drug recovered in urine after 5 days. In liposomal doxorubicin, 5% is recovered in 72 hours. After administration, doxorubicin is found primarily in the kidneys, small intestine, heart, and liver [Boulikas T. et al. Recent clinical trials using cisplatin, carboplatin and their combination chemotherapy drugs, Oncology Rep. 11:559-595 (2004)]. Side effects of doxorubicin can be significant, including cardiotoxicity, myelosuppression, and infertility. Other early-onset and dose related toxicities of doxorubicin also occur, such as nausea, chills, and pain on injection.

In the formulation of doxorubicin, a principal objective of the invention is to provide nucleic acid targeting to doxorubicin and to minimize the rapid absorption of doxorubicin to off-target sites. The rapid and broad distribution of doxorubicin is likely due in part, because of its small size and its tendency to rapidly bind within extra vascular tissues. Nucleic acid bound DOX can minimize rapid off-target binding and increase accumulation in metastasized and solid tumors. The saturation of DOX binding sites with nucleic acid residues causes reduction in off-target binding and enhances binding and co-transport of DOX with the carrier. Nucleic acid DOX carriers can be designed to promote increased circulation time, refractory uptake into primary and non-dividing cells, and preferential uptake into rapidly proliferating cancer cells.

Mitoxantrone is a synthetic, anthracenedione. Mitoxantrone, also known as Novantrone™, is indicated for metastatic carcinoma of the breast, myelogenous leukemia, adult non-lymphocytic leukemia, non-Hodgkin's lymphoma, and recently approved for the treatment of multiple sclerosis. Mitoxantrone is formulated at 2 mg/ml in sterile aqueous solution. In patients, mitoxantrone is distributed widely into tissues following intravenous administration and has a long elimination half life of 12 days. Side effects of mitoxantrone (MTN) include cardiotoxicity, bone marrow suppression, and hematological toxicity.

The drug loading of nucleic acid-mitoxantrone carrier (NAC-MTN) was assayed by column chromatography. Column data showed a single, concise peak corresponding to nucleic acid MTN complex at up to 50% weight:weight of mitoxantrone to nucleic acid carrier. At 70% MTN drug loading, complexing of the MTN to the nucleic acid carrier was less stable, producing a secondary, free drug peak.

In-vitro toxicity studies of nucleic acid-mitoxantrone carrier (NAC-MTN) were performed in MCF-7 breast cancer cells. In experiment (EX050404) a titration of the NAC-MTN was performed at different drug loading levels to determine if free drug was present. It was discovered that at 20% MTN loading in the carrier, the MTN was virtually 100% bound, with no more than 3% free drug. Furthermore, the total drug in the nucleic acid carrier was approximately 4× fold higher than the concentration required for 100% lethality, but showed equivalent toxicity to free MTN at about 30× fold concentration of MTN. Similar results with DOX were observed, but NAC-DOX showed no toxicity at a 2× fold the lethal DOX concentration.

In experiments designed to explore the differences in toxicity of nucleic acid-drug carriers delivered in different cell types, MCF7 breast cancer cells and COS7 transformed primate cells were treated with the nucleic acid mitoxantrone carrier. Differences in toxicity appeared to be related to the molar drug loading % in the nucleic acid. Different molar ratios of mitoxantrone (MTN) were loaded in genomic, double stranded DNA isolated from herring sperm (Promega) and incubated in cells for 72 hours. To test the effect of enhanced delivery, the NAC-MTN's were also formulated with a transfection agent and a lysosome disrupting agent, chloroquine at 50 uM. In the COS7 cells the toxicity correlated with the effective uM [MTN] in the nucleic acid. The higher [MTN] corresponding to higher drug:nucleic acid molar ratios and showed higher toxicity, likely because more drug was delivered to the cell per nucleic acid molecule. However, in MCF7 cells, the trend was reversed so that the highest [MTN] at the highest drug: nucleic acid molar ratio showed the least toxicity. Lower molar ratios of drug:nucleic acid were more effective against MCF7 cells. These surprising trends were highly repeatable, and repeated in samples treated with the transfection agent pPEG1020H or pPEG11105C.

In repeated studies, the SV40 transformed, COS7 primate cells showed higher susceptibility to the NAC-MTN than the MCF7 human breast cancer cells. In NAC-MTN at 9 uM (9 micro-molar), the toxicity in COS7 was greater than two times the toxicity observed in MCF7. Toxicity of the NAC in some cases depends on the molar ratio of drug to nucleic acid, with higher loading actually causing reduced toxicity. The reduced toxicity might be due to the tendency of MTN at high loading to cause a multiplex DNA particle which is more stable and less likely to release the drug or bind to a cellular target.

Nucleic Acid Targeting And Uptake

In general, the uptake of nucleic acids into cells is challenged by their polyanionic nature, macromolecular size, and the natural defense mechanisms of cellular membrane against entry of foreign nucleic acids. In order to effectively use nucleic acids as drug delivery molecules, sufficient nucleic acid-drug complex must be internalized by the cell to establish a therapeutic drug concentration.

The intrinsic properties of nucleic acids are advantageously utilized in the present invention in surprising ways. A principal advantage of the invention is that the drug can be inhibited from entry into a non-target cell because of an association with the nucleic acid. The inhibitory uptake is discovered to be cell selective and is based on many factors. In some embodiments, the masking effects are likely due to the poor cellular uptake of the nucleic acid-drug complex. As will become more apparent, many other advantages of the invention follow from controlling cellular entry processes.

Initial findings with nucleic acid-mitoxantrone showed that nucleic acid mitoxantrone complexes were ineffective against confluent MCF-7 cells. Since toxicity did not appear to increase proportionately with concentration of nucleic acid-drug, the lack of activity was initially thought to be due either to the low uptake of nucleic acid-mitoxantrone carrier by the MCF7 cells or to ineffective release of mitoxantrone from the nucleic acid. However, subsequent studies showed that freshly plated, non-established MCF7's (see figure) treated with nucleic acid-drug showed higher susceptibility to uptake and significantly higher toxicity. The data demonstrated the efficacy of the carriers but also showed that certain cell lines can be stimulated to uptake nucleic acid-drug carriers under specific conditions.

Uptake of nucleic acid-drug complex in different cell types under different conditions has been studied in an effort to determine the effect of drug loading, concentration, and incubation time on nucleic acid targeting. It was found that cell specific nucleic acid targeting was dependent on the drug loading w/w % and on the type of drug, or binding characteristics of the drug. Differences in uptake are likely due to the effect of drug loading on the structural shape of the nucleic acid-drug particle and the net charge. Higher drug loading generally resulted in greater differences in uptake between different cell types, with more significant differences occurring between faster and slower growing lines.

To compare the differential uptake of NAC-drug to the drug, the relative uptake and/or drug effects of the NAC-drug are normalized against drug alone. Normalization of NAC-drug to the drug corrects for experimental differences in cell density and intrinsic uptake factors related to the cell. In this way, the practitioner can measure the enhancement of targeting due to the NAC-drug complex and further optimize the composition to produce the desired targeting effects.

In one experiment, the toxicity of NAC-MTN complex at 12% w/w drug loading was assayed in four different cell lines, MCF7, COS7, 3T3, and BHK21 and compared with toxicity of MTN alone or MTN delivered with a cyclodextrin carrier (Example 10, EX42105). After 48 hours, significantly higher toxicity was evidenced in the faster growing cell lines COS7 and BHK21 than in MCF7 and 3T3. In MCF7's, NAC-MTN produced 33-35% reduction in toxicity compared with MTN only and CD-MTN. In COS-7's, NAC-MTN produced 17% reduction in toxicity compared with CD-MTN and 6% compared with MTN only. In 3T3's, NAC-MTN produced 27% reduction in toxicity compared with CD-MTN. In BHK21's, NAC-MTN produced 10% reduction in toxicity compared with CD-MTN but only 3% compared to MTN alone.

Subsequent experiments (EX51605, example 14) that compared NAC-MTN and MTN uptake in MCF7, COS7, 3T3, and BHK21 by fluorescence also showed enhanced uptake of NAC-MTN in faster growing cell lines compared to MTN alone. NAC-MTN prepared from either HSGDNA (genomic DNA) or phosphorothioated, single stranded DNA at 10% w/w, showed more substantial reduction in uptake in MCF7 and 3T3 than in COS7 and BHK21. Furthermore, NAC-MTN produced greater differential uptake in faster growing cell lines than a cyclodextrin-MTN formulation (CD-MTN).

Mechanistically, nucleic acid drug carriers operate very differently from current gene based drugs. Whereas, aptamers, anti-sense, and siRNA's have been proposed against targets within the body, the nucleic acid herein is used most preferentially to complex with the drug. Except in cases where sequence is important to binding the drug, the nucleic acid targeting aspects can operate primarily by sequence independent mechanisms. Highly effective nucleic acid based pharmaceuticals are shown herein that can be prepared from naturally occurring DNAs. In targeting, the selective entry is primarily an intrinsic property of the nucleic acid, due in part to cell membrane porosity, endocytosis processes and other factors.

In the present invention, a nucleic acid is used to bind a drug so that both entities are complexed together prior to administration. The nucleic acid serves as a nucleic acid carrier for the drug, such that it forms a non-covalent association with the drug. The nucleic acid can bind the drug entity by different mechanisms. Nucleic acid carriers that interact with drugs by intercalation and groove binding are highly useful in the invention and are disclosed herein. However, another highly useful class of therapeutic entities can be constructed by complexing a nucleic acid with a peptide or protein based drug. Nucleic acid-protein binding can be highly specific, depending on the sequence. Protein drugs, such as interferons are presently used in the treatment of hepatitis and other diseases. Recently, interferon has been conjugated to a PEG carrier (Nektar Pharmaceuticals) and licensed to Roche, N.J. The PEG-interferon conjugate showed significant improvement over interferon alone and was given fast track FDA approval in the treatment of hepatitis. The present invention can offer an efficacious alternative to PEG conjugation of interferon by utilizing a nucleic acid carrier that binds specifically to α-interferon. In NAC-interferon, the nucleic acid carrier is preferably a synthetic, double stranded or single stranded nucleic acid, of between 10 bp (base pairs) and 100 bp, or if single stranded, more preferably between 10 and 50 bases, and further comprises a nucleic acid sequence that binds interferon. The surprising activity of NAC-mitoxantrone and NAC-doxorubicin after intercalation indicate the potential advantages of NAC-interferon, i.e. that interferon can be bound but also available to a target within the body. However, there are several hurdles that must be overcome in NAC-protein drug, (i) retention of protein viability and activity, (ii) sufficient NAC-protein association to facilitate co-transport in vivo and (iii) the NAC-protein should have advantages over protein alone. The surprising and unexpected in vivo stability of NAC-mitoxantrone complex show, in principal, that NAC-interferon can sufficiently stabilize the nucleic acid against nucleases to facilitate co-transport in vivo. Furthermore, the in vivo as well as in vitro results of NAC-DOX and NAC-MTN show the potential for targeted delivery of proteins via the NAC-protein complex. Self-assembly of interferon with the nucleic acid can offer significant drug-masking and co-transport advantages, to reduce loss of interferon in the liver and enhance circulatory half life.

Nucleic acid targeting can also be highly useful in diagnostic applications, where it is desired to deliver a radio-diagnostic agent to a specific tissue within the body. Suitable agents used for diagnosis or treatment include radionuclides, technetium 99 m, iodine I-131, and phosphorus 32. Optionally, radio-diagnostic agents can be grafted to a suitable NABS to enhance binding to the NAC.

In an attempt to reduce the toxicity of drugs, many drug delivery strategies in the current art employ a polymeric carrier substance covalently attached to a drug. In general, the covalent conjugation of the drug to a polymeric carrier is more problematic, and less advantageous, due to problems of release [S. Papot, I. Tranoy, F. Tillequin, J. C. Florent and J. P. Gesson, Design of selectively activated anticancer prodrugs: elimination and cyclization strategies, Curr. Med. Chem. 2:155-185 (2002)]. Polymeric conjugated drugs, including pro-drugs, typically utilize enzymatic or pH labile bonds to selectively release a drug from the polymer. However, enzymatic lysis and release can be a complex process that is difficult to control in polymeric pro-drugs. For example, in the mAB-calcheamicin conjugate, Mylotarg ™, the hydrazone linker is unstable in physiological conditions resulting in approximately 50% of the drug existing in free form Bross et al. [approval summary, Clin Cancer Res. 7:1490-1496 (2001)]. Other mAB-drug conjugates containing doxorubicin, maytansinoid, and CC1065 have encountered similar problems of free-drug, linker instability and/or reduced drug potency [Doronina et al. Nat. Biotech. 21:778-784 (2003)]. Alternatively, if the covalent bond is too strong, the drug may not release, leading to an ineffective dose.

In contrast, NAC-drugs provide a novel means to effectively bind, co-transport, and mask the toxicity of drugs by non-covalent interactions between the drug and the NAC. The present invention provides an alternative means of association between a drug and a macromolecular entity, and more specifically the nucleic acid, wherein the drug remains in active form while non-covalently associated with the carrier. The invention is more versatile, in that many drug entities can be suitably incorporated in the invention, utilizing a single NAC. Thus, a large number of intercalating and groove binding drugs, as well as many other drugs that bind DNA, can be prepared as NAC-drugs.

A further impediment to current polymer based drug carriers is the unknown bio-compatibility of the polymer or of the linkage(s) used to conjugate the drug to the polymer. Linkage chemistries can introduce substantial background toxicity in drug-polymer conjugates (Parang et al., 2000). The introduction of synthetic polymers into the body has a certain element of risk and requires extensive safety screening. On the other hand, nucleic acid carriers have significant advantage over existing drug carrier polymers due in part to their intrinsic bio-compatibility, bio-degradability, non-immunogenicity, and safety. The cellular fate of nucleic acids in the body follow existing and natural pathways that are utilized in the continual elimination of cellular DNA.

In-vivo studies comparing treatment with NAC-MTN or MTN in mice, show surprising differences in the renal clearance and sub-organ distribution of NAC-MTN compared with MTN alone. In mice treated with repeated doses of either NAC-MTN or MTN, it was found that the effective MTN concentration in major organs such as the heart, lung, and liver was more than two-fold reduced in NAC-MTN treated mice than in MTN treated mice. Similarly, in mice treated with a single dose of NAC-MTN or MTN, MTN concentrations in NAC-MTN treated mice were significantly lower in all major organs including the heart, lung, liver, and kidney compared with MTN treatment alone. The clearance of MTN from blood correlated with decreased concentrations observed in major organs. Clearance of MTN from the blood was greater than four fold higher in NAC-MTN treated mice than in MTN treated mice. In studies designed to explore the stability and fate of the NAC-MTN complex in-vivo, MTN was shown to be effectively complexed with the nucleic acid carrier after clearance from the blood.

For certain drugs, it is desirable that the NAC-drug is not excessively absorbed into non-target tissues in order to prevent non-specific toxicity and to allow more drug availability to targeted regions. By reducing the non-specific absorption, lower doses can potentially be utilized, provided that circulatory half-life is sufficient to allow uptake in targeted cells or tissues. The in-vivo results demonstrate the significant potential of the NAC-drug complex for in-vivo targeting of a cytotoxic agent. The effective encapsulation of the drug, combined with the refractive uptake of NAC-drug into many of the normal functioning tissues of the mammal demonstrates that a significant fraction of the NAC-drug is available for targeting. Consequently, a factor that enhances uptake into a target will significantly enhance the nucleic acid targeting effect, since uptake and subsequent activity of the drug will occur to a greater extent in rapidly dividing cells such as proliferating tumor cells, virally transformed cells, and other cells or tissues with unique morphology. In other words, the reduction in non-specific absorption is an initial step in targeting, and can amplify the effect of other targeting entities that are optionally incorporated therein.

The complexing of a drug with the bio-compatible nucleic acid carrier is also shown to enhance the solubility of drugs that are unstable at physiological pH. This unexpected feature has advantages for the formulation and storage of many drugs in aqueous dosage forms. Doxorubicin and mitoxantrone are both precipitated at neutral pH in the presence of standard saline (HBSS or PBS). Formulation of these drugs with the nucleic acid carrier in phosphate buffered saline (PBS), Hank's buffered saline (HBSS), and DMEM media, was shown to prevent precipitation during storage.

Similar results were obtained for dipyridamole (DPA). Dipyridamole is a coronary vasodilator and functions primarily to reduce platelet adhesion and aggregation. Dipyridamole also inhibits adherence of cells by repressing the formation of fibrin. Dipyridamole for intravenous injection is given as a sodium chloride or dextrose injection with 5 mg of DPA, 50 mg PEG600, 2 mg tartaric acid, and water at pH 2.7. Dipyridamole is readily soluble in acidic solution, however at higher pH it can precipitate. Studies of the carrier with dipyridamole have shown that formulation with the nucleic acid carrier stabilized dipyridamole against precipitation in aqueous solutions at pH 7. This result suggests that the nucleic acid carrier can be used to enhance the in-vivo bioavailability of dipyridamole by preventing precipitation. Furthermore, the nucleic acid targeting of dipyridamole can provide unexpected therapeutic benefits by enhancing its accumulation in specific areas of the body.

Nucleic Acid Drug Carriers And Transfection Agents

Formulation of nucleic acid drug particles with other entities that bind DNA, such as transfection agents, can cause condensation and aggregation of the nucleic acid drug complex, forming nano-sized particles. In pharmaceutical preparations, highly preferred are micelle forming transfection agents, such as pegalated PEI, that can self assemble with the nucleic acid-drug complex to form PEG shielded, NAC-drug particles. The PEG shielded, nano-particles containing the nucleic acid-drug complex, are shown herein, to enter cells to a greater extent, due in part to a reduction in negative surface charge and enhanced transport across lysosomal membranes.

The combination of the nucleic acid-drug with a transfection agent, such as a micelle carrier, was found to significantly improve the performance of the NAC-drug against certain types of cells. For example, in studies with COS7 and MCF7 cells, nucleic acid-drug carriers that are complexed with micelle carriers show higher cytotoxic activity, presumably due to greater uptake of the nucleic acid-drug carrier. In the formation of nucleic acid-drug with a transfection agent, the nucleic acid is first combined with the drug to form the nucleic acid-drug complex. The transfection agent is then combined with the nucleic acid-drug particles to form a micelle particle. In the assembly of nucleic acid-drug carriers, the order of addition is important. It was discovered that addition of the drug after complexing the DNA with the transfection agent prevented the drug from effectively binding the DNA and caused higher toxicity in cell culture due to the presence of free drug. The suitability of a transfection agent must also be determined based on the binding properties of the drug, since some transfection agents may cause displacement of the drug and liberation of free drug in solution by competitive binding.

The transfection agent can be a cationic polymer, lipid, liposome, micelle, or other suitable agent that enhances uptake of the nucleic acid-drug particles. Cationic polymers containing positively charged amine groups, such as polyethylenimene (PEI's), poly-arginines, and polyamidoamines, can condense the nucleic acid-drug particles into torroidal shapes, typically increasing the size by incorporating many nucleic acid-drug molecules into a single aggregate. Cationic lipids, such as the well known Lipofectamine™, DOTMA, and others, are also known to form nano-sized particles with DNA due to association of quaternary ammonium groups. Highly preferred are naturally derived polyamines such as the protamines, spermines, and their derivatives. The cationic polymer is preferably combined with the nucleic acid-drug complex at a nitrogen to phosphate ratio (N/P) that is sufficient to provide optimal transfection conditions with minimal non-specific toxicity.

The incorporation of transfection agents in the present invention allows the practitioner to control the relative degree of cellular entry, thereby modulating the various NAC-drug effects and controlling the uptake of drug into an extra vascular target. In-vitro experiments (EX021605) have demonstrated that formulation of the nucleic acid-drug in a pendant polyethylene glycol-polyethylenimine (pPEG-PEI) shielded micelle can significantly enhance uptake, particularly in cells which already show preferential uptake. In some cases, combination of transfection agent with NAC-MTN did not significantly enhance toxicity against confluent, breast cancer cells. Without limiting the invention, it is hypothesized that when the MCF-7 cells have reached confluency, their rate of division is significantly slowed, and they show very low susceptibility to the NAC-MTN carrier, with or without the transfection agent. However, when the experiment was repeated with the same cells plated at low density, the rate of uptake was significantly enhanced for the NAC-MTN, and was further enhanced for the NAC-MTN formulated with the transfection agent pPEG1020H (KKBIOMED INC.), as shown in the disclosure (EX112304).

The surprising and unexpected in-vitro results of NAC-drugs delivered with a transfection agent show the potential for similar enhancement of nucleic acid targeting of drugs in-vivo. Transfection agents can enhance NAC-drug accumulation in rapidly dividing cancer cells, while at the same limiting uptake in non-dividing and primary cells. Without limiting the invention, the magnifying effect of a transfection agent is likely due to rate limiting processes in a cell that effect uptake of the NAC-drug. Thus, in cells that are already refractory, transfection agents will show proportionately less improvement on uptake of NAC-drug, whereas in cells that are permissive, the uptake of NAC-drug can be enhanced. In general, methods which enhance the delivery of nucleic acids across the cellular membrane are therefore expected to provide benefits, particularly in-vivo, since the uptake in target cells will be enhanced but toxicity to normal healthy cells will be minimized.

It is shown in-vitro (EX032505) that the NAC-drug can be highly effective against cells which are in suspension, since cells show a disproportionately higher uptake than when they are adhered to a surface. The in-vitro results suggest that the NAC-drug may have greater efficacy against proliferating cells which propagate via the blood stream, but also non-adherent types, since these cells may uptake the NAC-drug to a greater extent than primary, adherent cells.

Transfection agents can be used in conjunction with the present invention, to target the nucleic acid-drug carriers to specific organs within the body. For example, nucleic acids which are condensed by cationic polymers, are shown to accumulate primarily in the lung or the liver depending on the surface charge of the particle [Mahato et al. J. Pharm Sci. 84:1267-1271 (1995 )]. Transfection agents such as polyethylenimine, coupled to targeting moieties such as transferrin, galactose, mannose, galactosylated polylysine, membrane transduction peptides, and folic acid, among others, are also known in the art, and can be used in conjunction with the present invention to provide additional localization and targeting of the nucleic acid-drug particles [Wagner et al., Pharm Res. 21:8-14 (2004), Kursa et al. Bioconjug. Chem. 14:222-231 (2003) and Kircheis et. al. Gene Ther. 8:2840 (2001)].

In cationic liposomal delivery, it was shown that when an ODN was complexed to a cationic lipid to form a lipopolyplex particle, the resulting particles more selectively targeted monocytes and showed the lowest uptake in T cells [Kronenwett R., Steidl U., Kirsch M., Sczakiel G., Haas R., Oligodeoxyribonucleotide uptake in primary human hematopoietic cells is enhanced by cationic lipids and depends on the hematopoietic cell subset, Blood, 91:852-862 (1998)].

Transfection agents utilized herein, can provide significant advantages to the delivery of NAC-drug complexes to the cell. The transfection agent is utilized to modulate the delivery of the NAC-drug complex, thereby influencing its pharmacokinetic activities in many surprising ways. Unlike conventional nucleic acid based drugs, NAC-drug complex can impart more potent effects, and the transfection agent can amplify these effects due to enhanced uptake of NAC-drug polyplex, across the cellular membranes, and also by novel intracellular trafficking of the NAC-drug polyplex micelle.

NAC's With Nuclear Localization Sequence (NLS)

The nucleic acid sequence can be designed to enhance the translocation of the carrier across the cellular membrane, to improve pharmacokinetic properties, or to facilitate nuclear transport. Certain nucleic acid sequences, or promoter sequences, derived from viruses such as SV-40, can improve the intracellular transport of plasmid DNA to the cell nucleus [Dean D. A,. et al., Effect of a DNA nuclear targeting sequence on gene transfer and expression of plasmids in the intact vasculature, Gene Ther. 10:1465-1470 (2003)]. NAC-drugs comprising a plasmid DNA with a drug payload, can provide unique intracellular trafficking of the drug and novel drug function.

NAC Aptamer

Nucleic acid ligands or aptamers, are nucleic acids that can bind to another molecule in a sequence specific manner, said binding referred to herein as “aptamer binding”. Previously, aptamers have been proposed to bind endogenous targets, but their utility has been limited by problems of cellular uptake, inactivation by nucleases, and ineffective hybridization. In the present invention, aptamer binding provides a novel means of non-covalent association between the NAC and the drug. NAC aptamer-drug complex can provide effective drug-masking and nucleic acid targeting of drugs but also novel intracellular release and transport of a drug within the cell. Once inside the cell, modification of the NAC aptamer structure can cause efficacious release of the drug. Bi-functional NAC aptamers comprising a drug binding region or aptamer and a targeting aptamer advantageously combine passive targeting and active ligand targeting in a single particle. For example, NAC-drug's further comprising a nucleic acid sequence that binds an endogenous protein, such as a cellular surface protein that is unique to a tumor, can readily accumulate at the tumor site and increase the concentration of a drug at the target. Aptamers that bind specifically to cellular surface proteins and other proteins, such as HIV reverse transcriptase, HIV rev, PDGF, thrombin, VEGF, and vasopressin, among others, have been identified [Roberts et al., Curr. Opin. Struc. Bio. 9:521-529 (1999)]. Conjugation of a drug to an aptamer specific for platelet derived growth factor (PDGF) has been proposed (Stanton et. al. US pat. App.# 20040249130). The present invention provides the following substantial advantages over prior art; 1) the drug is not chemically modified, 2) the NAC-drug particles self-assemble in aqueous media, 3) drug toxicity is significantly reduced by NAC-drug complex, and 4)NAC-drug complex can enhance uptake of the nucleic acid across cell membranes. In general, conjugation of the drug to the aptamer is less preferred since it requires chemical modification of the drug and can result in reduced drug efficacy or ineffective release. Drug conjugation can also lead to inhomogeniety, premature release of drug by plasma esterases (Parang et al. p109 2000), and similar difficulties encountered with other polymer-drug conjugates. Formation of microspheres in organic solvents described by Farokhzad et al. can be impractical for water soluble drugs, potentially leading to drug inactivation, ineffective drug masking, ineffective uptake into tumors, or rapid release. Formulation of a drug in microspheres, such as the poly-lactic acid microspheres of Langer et.al. [Farokhzad O. C. Langer R., et al. Cancer Res. 64:7669(2004)] are impractical since they can inhibit the formation of DNA-drug complex by creating steric interference or charge repulsions between the drug encapsulated particles and the nucleic acid. Moreover, drug conjugation by Stanton et al. “teaches away” from the present invention since grafting of the drug can interfere with aptamer-drug complexing, thereby circumventing the drug-masking qualities and other advantageous properties of the NAC-drug complex.

NAC aptamers are particularly useful in forming NAC-drug composition with many kinds of drugs such as taxoids, antibiotics, peptide drugs, among others, that ordinarily lack suitable affinity with nucleic acids. Nucleic acid sequences that are appropriate for binding a drug are prepared from aptamer libraries utilizing a reiterative process as disclosed in U.S. Pat. No. 5,723,594 or US Pat. App. #2005037075, hereby incorporated as reference. Due in part to the very strong and highly specific binding of an aptamer with a protein, aptamers are especially applicable to the preparation of NAC-protein drugs. In one example, NAC-protein drugs are optionally conjugated via the nucleic acid to a suitable masking agent such as a PEG, to form long-circulating PEG-NAC-protein drugs that resist clearance from the kidneys. Moreover, bifunctional NAC's comprising two distinct protein binding sequences or domains, can bind a therapeutic protein with one sequence and target another protein, such as a cell surface receptor, with the other sequence. The sequence specificity in bi-functional aptamer NAC∝s can be advantageous in localizing the drug at one end of the nucleic acid, thereby minimizing the potential of interference with the targeting end.

NAC's Conjugated To Masking Agents

Also suitable in the invention are nucleic acid conjugates, in which the nucleic acid-drug carrier is optionally grafted to a “masking agent”. A suitable masking agent is optionally selected from a polyethylene glycol, HPMA, polyglutamate, poly(lactate), human serum protein, polysaccharide, lipid, fatty acid, alkyl chain, or a suitable biocompatible polymer derivative thereof. Suitable fatty acids are oleic acid, palmitic, stearic, among others. Suitable lipids are amphiphilic molecules that comprise a hydrophilic and hydrophobic end and include without limitation phosphotidylcholine, phosphatidylethanolamine, phosphotidylcholine derivatives, cholesterol, hydroxy-cholesterol, and derivatives. Suitable saccharides include polysaccharides, oligosaccharides, cyclodextrins, dextrans, inulin, and others. In this invention, the nucleic acid serves to bind the drug, while the masking agent provides additional pharmacokinetic properties. Many conjugates of anti-sense ODN's and siRNA's with polyethylene glycol, cholesterol, and other ligands have been described in the art and methods of synthesis are incorporated herein by reference [Soutschek et.al. Nature 432:173-188 (2004), Jeong et.al. J. Con. Rel. 93:184 (2003), Wang et al. Antisense And Nuc. Acid Drug Dev. 13:169-189 (2003)]. Incorporation of masking agents in the invention can impart many favorable and advantageous properties to the nucleic acid-drug complex such as prolonged circulation, uptake across the blood brain barrier, resistance to nucleases, masking against immunological factors, among others. Mono-functional and bi-functional PEG's that have functional carboxylic acids, hydroxyls, N-hydroxy succinimide (NHS) ester, epoxy, isothiocyanate, among others, are available from commercial suppliers such as Sun Bio Inc and Nektar Therapeutics Inc., and can be utilized in the synthesis of NAC-PEG conjugates. The DNA can be conjugated to the masking agent by a variety of means. Synthetic nucleic acids with a reactive primary amine can be synthesized with DNA synthesizers using standard phosphoramidate chemistry or obtained directly from many commercial suppliers. Methods are also commercially available that can be used to incorporate an amino labeled nucleotide into native DNA using an appropriate enzyme (Invitrogen, Carlsbad Calif.). The nucleic acid-amine can then be conjugated to a suitably derivatized N-hydroxy succinimide ester (NHS) PEG, NHS-oleic acid, etc. under aqueous conditions at pH 7. Ligands that have carboxylic acids are also readily grafted to the amine functionalized DNA by use of a carbodiimide catalyst. In the exemplary disclosure, a 5′ amine labeled ODN is conjugated to a pendant PEG propionic acid via catalysis by 1-ethyl-3-(-3-dimethylaminopropyl) carbodiimide (EDC). In the invention, the nucleic acid is preferably conjugated to the masking ligand in such a way that the nucleic acid binding to the drug is not impeded.

Nucleic acid carriers can significantly enhance the in-vivo safety of many kinds of drugs due to the high biocompatibility of the carrier combined with effective drug masking and stability. Although the NAC-drugs are highly suited to parenteral routes, the ideal mode of administration will likely be determined by a number of factors, such as the circulatory half life, plasma steady state level, non-specific uptake, uptake into the target site, etc. Slow release of the NAC-drug from a patch or hydrogel may improve the therapeutic value in certain applications. Charged nucleic acid-drug carriers can be delivered transdermally via electroporation, also known as iontophoresis. Gel migration studies of nucleic acid-mitoxantrone complexes show that mitoxantrone can be loaded at 10% weight to weight of nucleic acid carrier and effectively transported through an agarose gel in an electric field. The nucleic acid-mitoxantrone complex was shown to co-migrate with the mitoxantrone at a rate that was approximately three times the gel migration speed of mitoxantrone alone. In the presence of no carrier, mitoxantrone normally migrates in the opposite direction due to its positive charge. In an electric field, nucleoside drugs are shown to co-migrate with the NAC in the presence of 4′, 6 diamidino-2-phenylindole (DAPI). The data show the potential of nucleic acid-drug carriers which can deliver highly effective drug cargoes to a subdermal location. Multi-drug resistance (MDR), is currently a significant problem in the treatment of cancer. The novel properties of nucleic acid-drugs can be used to overcome multi-drug resistance by delivering drugs via alternative pathways that are difficult for the cell to circumvent. Broad resistance to small molecule therapies is reportedly influenced by membrane P-glycoprotein, or P-glycoprotein, encoded by the MDR1 gene, that pump small molecules out of the cytoplasm [Hendricks C. B. et al, Effect of P-glycoprotein expression on the accumulation of cytotoxicity of topotecan, a new camptothecin analogue, Cancer Res. 52:2268-2278 (1992)]. Because the entry of nucleic acid-drugs into a cell is primarily an endocytotic process, the nucleic acid-drug carrier is transported via lysosomal compartments, allowing it to by-pass the cytoplasm and avoid being pumped out of the cell. Changing the pathway, and enhancing targeting, can have potential benefits to the treatment of cancers that are refractory to small molecule therapies.

Delivery Of Nucleoside Drugs

Nucleoside drugs are an important drug class that are currently used to treat HIV, hepatitis and cancer. The anti-HIV drug, AZT (Retrovir™), is a thymidine nucleoside with an azido function on the 3′ end of the deoxyribose ring. Ribavirin™ and Viramidine™ (a ribovirin analogue) have shown significant efficacy in treatments against hepatitis C. Nucleoside drugs can significantly benefit from nucleic acid targeting by reducing drug accumulation in bone-marrow and in red blood cells. Viramidine™ is positively charged, and can associate effectively with the negatively charged phosphates of the nucleic acid carrier. To enhance systematic targeting and uptake of Viramidine™ into the liver, the NAC can be further conjugated to liver targeting ligands [(Bartholomew et al, J. Viral Hep. 2:273-278 (1995)]. In NAC-AZT, the association of AZT with NAC is optionally enhanced by first conjugation of AZT with the intercalator 3,6 diaminoacridine to form an enzyme labile, ester linkage, as shown in the disclosure.

In other aspects, it was discovered that nucleoside drugs can be co-delivered with DNA by non-covalent incorporation of a nucleic acid binding spacer (NABS), such as 4′, 6 diamidino-2-phenylindole (DAPI). DAPI was unexpectedly found to bind with both AZT and phosphorylated nucleosides. Other studies with diaminoacridine (DAA), showed similar binding of DAA with nucleosides. Without limiting the invention to a specific mode of action, the DAPI forms an effective association between the DNA and the nucleoside, due to mutual affinity. The complex between DAPI, nucleoside triphosphate, and DNA was shown by comparing co-migration patterns of DNA-DAPI and DNA-DAPI-nucleoside in gel electrophoresis. In fluorescence assay, DAPI was shown to bind more effectively to the nucleoside in the presence of DNA, but only weakly interacted without DNA, suggesting that DAPI was binding within the nucleoside-DNA complex. These results show that NAC-AZT drugs, among others, can be prepared by incorporation of the appropriate NABS at sufficient ratio to provide association between the nucleoside drug and the NAC.

NA's Conjugated With Targeting Moieties (T M)

The incorporation of certain targeting moieties in the invention can further enhance the nucleic acid targeting and other features of the invention. Certain molecules such as folic acid, are known to be selectively uptaken into a cell by their recognition at a cell surface receptor. Targeting moieties are preferably grafted to the nucleic acid carrier. Targeting moieties that are suitable in the present invention include fucose, galactose, mannose, steroids, folic acid, peptides, peptide derivatives, transferrin, oligoarginines, peptide fragments, viral fragments, antibodies, antibody fragments, and steroids and are reviewed by D. Lochmann et.al. “Drug delivery of oligonucleotides by peptides” J. Pharm. and Biopharm. 58:237-251 (2004). Nucleic acid carriers with targeting moieties that target hepatocytes can accumulate selectively in organs such as the liver to provide therapeutic benefit.

Membrane transduction peptides (MTP) are peptides that have the capacity to enter a cell due to recognition at a cell surface receptor or “destabilization” of the cell membrane. In other embodiments, the MTP is optionally grafted to the nucleic acid carrier to usefully enhance the uptake of the NAC-drug into the cell. Synthetic cationic peptides containing arginine and lysine such as GALA and KALA, can be useful to complex with negatively charged nucleic acids. More preferred are naturally occurring cationic polyamines such as protamine and pPEG-protamine, shown herein to effectively deliver NAC-drug complex to cells. Amphiphilic E5CA sequence was used by Pichon et al. to deliver ODN to a cell. MTP have been less effective for delivery of anti-sense ODN, possibly owing to the lack of lysosome release. Useful MTP peptide sequences include GALA (Szoka et al. and Parente et al. 1988), KALA (Wyman et al 1997), Melittin and EFCA (Midoux et al 1995), JTS-1 (Gottschalk et al. 1996), FLUOS (Oehike et al. 1998), RGD (Erbacher et al. 1999), HA (Lear et al. 1987), INF1 and INF7 (Plank et al. 1994), E5 and K5 (Murata et al. 1991). Other useful peptides derived from viruses include TAT (HIV derived), Ant (Drosophila), SV-40 (Simian virus 40), and nucleoprotein (influenza virus).

Recently, antibody-drug conjugates have been utilized for targeting drugs. One disadvantage of antibody-drug conjugation is the problem of insufficient drug release from the antibody once the antibody-drug reaches the target site. This problem is avoided in the present invention by conjugating the antibody directly to the nucleic acid carrier. The suitable drug is complexed with the mAB-nucleic acid to form a mAB-NAC-drug complex that can actively target and release a drug to a cell.

Like antibodies, aptamers have also been proposed to target cellular antigens. One of the difficulties in aptamer targeting is co-transporting and releasing the drug once it reaches the target. In Farokhzad et al. US pat app.# 20050037075, aptamers are covalently coupled to poly(lactic acid) microspheres. Formulation of a drug in the microspheres is less practical since it requires treatment with organic solvents and drug can rapidly dissociate from the particles once it is administered, diminishing the targeting effect. Conjugation of the drug to the aptamer ligand is also less practical, due to similar problems of antibody-drug conjugates previously described. These difficulties are overcome by complexing the suitable drug with the nucleic acid to form a nucleic acid-drug complex. The effective complexing between the drug and the nucleic acid ligand can be enhanced by selection of drug or suitable nucleic acid with a suitable sequence, or more preferably an aptamer sequence that is specific to the drug. One of the difficulties of utilizing aptamers as targeting ligands is the potential for inhibition of the ligand, for example, due to binding of the drug. This problem can be overcome by utilizing a bifunctional nucleic acid that comprises cell surface targeting end (aptamer) and a drug binding end (aptamer). Bi-functional NAC's can be highly advantageous to form NAC-complex that binds the drug but also leaves the cell targeting aptamer unimpeded.

Dual Functioning NAC-Drugs

The use of short chains of nucleic acids and even monomers comprising nucleosides, can be efficacious in preparing the NAC-drug complex. It was discovered that nucleosides and nucleotides form complexes with DNA binding agents, including intercalators and minor groove binders. With fluorescent drugs, the binding can be observed as a fluorescent enhancement or shift. Enhanced binding and fluorescence shift was observed with nucleotides due to the positively charged nitrogens on the drug interacting with negatively charged phosphates. NAC's composed of nucleosides, nucleotides, derivatives, or polymers thereof, are highly useful and can be utilized in the binding of DNA interacting drugs to form NAC-drug complex. Furthermore, when the NAC is a bio-active nucleoside or more preferably a bio-active nucleotide, the NAC-drug complex forms a dual functioning entity, with multiple drug action, thereby facilitating enhanced therapeutic benefits in the treatment of a malady. Additionally, NAC-drugs that are dual acting against a target by independent, bio-active mechanisms can potentially overcome resistance (MDR) by alternative pathways of action, thereby overwhelming the cancer cell or virus. Furthermore, NAC-drugs with multiple agents can have synergistic drug effects, since both agents are delivered simultaneously to an address.

NAC-drugs comprising a bio-active nucleotide drug are particularly advantageous over current nucleoside drugs and nucleoside pro-drug conjugates. Since nucleoside drugs must be phosphorylated prior to becoming active in the cell, nucleotide drugs are more advantageous and faster acting, because they by-pass an activation step. However, the usefulness of nucleotide drugs as chemotherapeutics have reportedly been limited by rapid dephosphorylation at the cell surface and by poor cellular uptake [Parang et al., Curr. Med. Chem. 7:1010(2000)]. Attempts to conjugate nucleotide drugs, such as AZT pro-drugs, have encountered problems with linker chemistry or low uptake that have reduced drug potency. In contrast, these problems can be avoided by complexing a nucleic acid binding drug with the nucleotide drug to reduce the negative charge of the particle and facilitate transport across cellular membranes. More preferably the drug contains one or more nitrogens that can associate effectively with the phosphates of the nucleotide. Enhanced nucleotide-drug binding is accomplished by using intercalators with quaternary nitrogens. It was found that the nucleoside-drug complex are sensitive to addition of phosphate salts, which likely disrupt the complex by competitive binding. Interference by dilute potassium phosphate was not as significant in nucleotide-drug complex. Complexing between the drug and the nucleotide can reduce activity of the drug in the blood stream as well as interfere with phosphorylating enzymes that can inactivate the nucleotide prior to uptake at the target site. Furthermore, nucleotides combined with the drug at appropriate N/P ratio, will target tissues by reducing entry of the drug into cells that are refractory, and enhance accumulation in cells that are permissive. The nucleotide-drug complex, is an effective NAC-drug complex in which both the NAC and drug have biological function to simultaneously target and induce potent cellular effects.

Summary

Nucleic acid drug carriers have significant potential in the treatment of cancer and many other diseases. Currently, there is an un-met need for enhanced targeting of therapeutic agents, particularly cytotoxic agents used in the treatment of cancer. Cancer is still a leading cause of morbidity and new treatments are required that are more efficacious and have fewer side-effects. It is shown herein that nucleic acid drug compositions utilizing nucleic acid targeting have significant advantages over existing art and are capable of inducing potent effects on cells by novel mechanism of action. Nucleic acid targeting can reduce the non-specific activity of therapeutic agents and enhance their accumulation in target areas of the body through novel delivery and release mechanisms. In-vitro, nucleic acid drug carriers are shown to selectively concentrate in cells during active cellular growth and division, making said agents potentially useful in treating aggressive and metastasized cancers. Nucleic acid drug complex are shown to preferentially target cells based on cell type, and more readily accumulate in cells that are more permissive to uptake, such as rapidly proliferating cells and transformed cells. Furthermore, the practitioner can enhance nucleic acid-drug targeting by inclusion of one or more transfection agents, or optionally a transfection agent coupled to a cell targeting moiety. The effective association formed between the drug and the bio-compatible nucleic acid carriers provides a novel means of linking drugs with targeting moieties such as folic acid, membrane transducing peptides, antibody, or a nucleic acid ligand, to provide active targeting of the NAC-drug to a site within the body. The NAC-drug complex is advantageous to enhance the uptake of the nucleic acid carrier and/or nucleic acid ligands across cell membranes. Nucleic acid drug carriers can potentially circumvent cellular mechanisms that convey multi-drug resistance, by novel intracellular uptake and transport pathways. Nucleic acid carriers can provide numerous other useful properties to enhance the in-vivo stability, solubility, and bioavailability of drugs, thereby enhancing a drug's therapeutic index. The use of nucleic acid as a bio-compatible drug carrier represents a novel application of a molecule that continues to revolutionize biology and now the pharmaceutical sciences.

### EXAMPLES OF THE BEST MODES FOR CARRYING OUT THE INVENTION

### Example 1

Determination of drug loading limit of mitoxantrone (MTN) in a nucleic acid carrier (NAC) using a 2.5×9 cm G25 column (EX031904) and herring sperm DNA (HSDNA approx. range 10-100 bp Sigma-Aldrich). Eluent was 0.025M KP buffer pH 6.85 in 25% MEOH. Run#1 (10% drug loading) combined 0.968 mg MTN (0.1 ml stock) with 2 ml H2O. Added 10 mg (2×128 ml of 39.1 mg/ml) 318A HSDNA carrier. Eluted on column, a single band was observed eluting at 15-20 ml total volume. Collected 5 ml and checked absorbance scan of 0.2 ml sample on Synergy HT1. Run#2 0.484 mg MTN was dissolved in 2 ml and eluted on column. The MTN bound to the top of the gel bed. Run#3 (20% drug loading) 0.968 mg MTN with 2 ml H2O and 5 mg 318A carrier and elute on column. Peak was very concise, eluted between 16-20 ml. Collected 7.5 ml and read absorbance scan of 0.2 ml sample. Run#4 (30% loading) Combined 0.1ml 9.68 mg/ml MTN with 1 ml H2O, followed by 0.064 ml 39.1 mg/ml 318AHSDNA carrier, which formed a precipitate. Sonicated 2 min. low power to produce clear, blue solution. Eluted on column, formed concise band eluting at 16-20 ml total volume. Run#5 (50% loading) Combined 0.015 ml or 0.587 mg 318A HSDNA with 3 ml H2O. Added 0.05 ml of 10.3 mg/ml MTN slowly. MTN solubilized readily until the last 10 ul was added, which immediately caused ppt. of MTN. Addition of 0.015 ml or 0.587 mg 318AHSDNA and brief mixing produced a clear solution at 50% loading. Added 1 ml eluent to the solution and eluted the entire vol. on column. Produced concise band eluting between 16-20 ml. Run#6 (77% loading). Combined 0.015ml 318A carrier (0.587 mg) with 3 ml H2O. Slowly added 45 ul or 0.45 mg MTN to give 77% w/w. Elution on column produced a concise band. However, after several minutes the band fragmented and a free MTN band was observed.

TABLE 1
Determination of drug loading limit
of NAC-MTN using G25 column

| Drug Loading % | G25 Column Results |
|---|---|
| Drug only | Single band, slow |
| 10 | single, concise band, fast |
| 20 | single, concise band, fast |
| 30 | single, concise band, fast |
| 50 | single, concise band, fast |
| 77 | Two bands, slow and fast |

### Example 2

Showing release of drug from the NAC carrier, using S100 column (EX051904) Column S100H.R. (high resolution S100, Pharmacia) 2.5×9 cm, eluent 25% MEOH, 25 mM KP pH6.85. Combined 9.5 mg 428A HSDNA with 0.47 mg MTN in 1 ml of eluent and eluted on gel. Most of the MTN was separated as a blue band that propagated very slowly. A DNA peak was detected by absorbance A260 at about 46 ml total elution, indicating that the DNA and MTN were separated by the column. Addition of 100 mg of a BCD-SBE 201A, to the column bound to the MTN band which caused rapid propagation and tightening of the MTN band due to complexing with the BCD-SBE201A (beta-cyclodextrin polymer). This example demonstrates the principal of release of drug from the NAC carrier.

### Example 3

Toxicity of NAC-MTN or NAC-DOX, at 50, 16.9, 5.6, and 1.9 ug/ml final drug concentration and 5.1 % drug loading in human MCF-7 cells. Sample A: 15 mg of HSDNA (EX031804A) was combined with 0.76 mg MTN(mw 517.4) or (sample B) 0.76 mg DOX (mw 579.99) to give 152 ug/ml final drug and 5.1% loading in 5 ml final vol. Samples C, D were MTN, or DOX only controls at equivalent final [drug] without HSDNA. Sample E was HSDNA, no drug and sample F was H2O only. MCF-7 cells were trypsinized and plated out previously. Triplicate samples were diluted 0.1 ml in 0.2 ml 1× media to give 3× fold serial dilution in 0.2 ml final. After 16 hour incubation the cells were visually inspected for signs of toxicity. Toxicity was scored as the following: 50%=one half all cells rounded, un-adhered or crenated.

TABLE 2
Toxicity of NAC-MTN and NAC-DOX at
fixed % w/w and varying [drug]

| Sample | 50 ug/ml | 17 ug/ml | 5.6 ug/ml | 1 .9 ug/ml |
|---|---|---|---|---|
| A | no tox. | no tox. | no tox. | no tox. |
| B | no tox. | no tox. | no tox. | no tox. |
| C | 100% | 100% | 90% | 20% |
| D | 100% | 80% | 20% | 10% |
| E | no tox. | no tox. | no tox. | no tox. |
| F | no tox. | no tox. | no tox. | no tox. |

The NAC showed significant protection of the cells from harmful effects of the drug, even at very high effective drug concentration of 50 ug/ml (97 uM and 86 uM for MTN and DOX respectively). This was a surprising result since free MTN produced 90% toxicity in group C at only 5.6 ug/ml. Similarly, DOX alone produced 80-90% toxicity at 17 ug/ml in group D.

### Example 4

Correlation between drug loading and toxicity of NAC-MTN or NAC-DOX at fixed [drug] of 0.15 mg/ml or 0.3 mg/ml respectively, against MCF7 cells. (EX042804).

Mitoxantrone (MTN, Sigma Aldrich) and Doxorubicin HCl (DOX) were diluted in HBSS to 1 mg/ml final. DNA, from degraded herring sperm DNA <40 bp (HSDNA, Sigma Aldrich), was prepared in water at 10 mg/ml. The drug was diluted in water or HBSS first, followed by addition of the DNA, in order to prevent precipitation. Precipitation of drug was especially a concern at 40% loading of MTN, which is the loading limit for HSDNA (degraded) and so the DNA is added slowly to the diluted MTN (the reverse addition leads to precipitation). All NAC-MTN formulation stocks were prepared at 0.15 mg/ml final MTN, and different concentration of DNA in order to give 15, 25, and 35.7% final drug loading w/w % of MTN/DNA in 0.5 ml final volume. All NAC-DOX formulations were prepared at 0.3 mg/ml final DOX, and either 5, 10, or 15% loaded drug weight/weight % of DOX/DNA in 0.5 ml final volume.

MCF-7 cells were previously dispersed and grown under 5% CO2 in standard 96 well tissue culture plates to confluency. All NAC-drug formulations were diluted 5× by adding 0.05 ml sample to the plate followed with 0.2 ml 1XDMEM (EX409media) per well. Final MTN drug was 0.03 mg/ml or 0.058 mM and final DOX was 0.06 mg/ml or 0.103 mM in cells. The NAC-drug and drug only samples were then incubated with the cells for 72 hours and assayed by MTT viability assay.

TABLE 3
The percentage loading of each NAC-MTN or NAC-DOX formulation
and correlating toxicity in MCF7. The 0% loading corresponds
to drug only, without carrier.

| NAC-MTN | | | | |
|---|---|---|---|---|
| % loading | 15 | 25 | 35.7 | Drug only |
| % Tox | 21 | 30 | 43 | 79 |
| NAC-DOX | | | | |
| % loading | 5 | 10 | 15 | Drug only |
| % Tox | 21 | 65 | 62 | 67 |

### Example 5

Cell culture assay for unbound drug in NAC-MTN or NAC-DOX. The [NAC-drug] was increased at different w/w % to detect presence of free drug in a given NAC-drug formulation. The correlation between cell toxicity, [NAC-drug], and % w/w were used to determine the presence of free drug. (EX050404) .

MCF7 cells were seeded and grown to confluency in a standard 96 well tissue culture plate. MTN and DOX were diluted in HBSS to 1 mg/ml and 0.4 mg/ml stocks respectively. Drug was combined with appropriate amount of HSDNA 10 mg/ml and/or 1× DMEM media to give four stocks at 1, 2, 4, and 8× concentration of DNA/Drug, at fixed mass %, and a 1 ml final volume. A concentration series was prepared for each drug/DNA mass % of 5%, 10%, and 20% for NAC-MTN and 2, 4, and 8% for NAC-DOX. Controls, DNA only, and MTN or DOX only were also prepared. The NAC-drug was then diluted at 2:1 with 1× DMEM media to give a ⅔ fold dilution and 0.3 ml was added to triplicate wells containing confluent cells. For the MTN plate rows 2,3,4 were reserved for 5, 10, 20% loaded DNA, row 5 was DNA only and row 6 was MTN only. In the DOX plate, rows 2, 3, 4 were reserved for 2, 4, and 8% DOX loaded DNA's, row 5 for DNA only, and row 6 for DOX only. Cells were incubated with samples for 72 hours in a cell culture incubator at 5% CO2, and then assayed for cell viability by MTT assay. For each sample, toxicity was plotted against the drug concentration. Surprisingly, the NAC-MTN at 20% drug loading, showed no toxicity at about 4× the [drug] required for 100% toxicity and produced about a four fold reduction in toxicity relative to MTN only, or a 400% reduction. Similarly the NAC-DOX produced about a 2× fold reduction in toxicity relative to DOX alone or a 200% reduction.

### Example 6

Solubility Enhancement of Dipyridamole with NAC.

Dipyridamole (DPA, m.w. 504.6) was prepared as 10.27 mM (52.9 mg/ml) solution in water acidified with HCl. DNA was from HSDNA 428A) 10 mg/ml in H2O prev. heated to 80C.

Prepared samples 1-3 by combining 25 ul of DPA and 0.2 ml DNA to give a turbid solution. Added 7 ul of 1M HCl to clarify, followed by 0.1 ml of 1M potassium phosphate buffer (KP) pH7. Repeated for samples 4-6 and replace DNA with water. Let solutions stand 16 hours, more precipitate was observed in drug only samples than in NAC-DPA complex. Read absorbance of 0.3 ml supernatant at A410. Higher values corresponded to more drug solubilized. Using the approximation that half the drug produced about half the absorbance, the NAC increased drug solubility by up to 56% over saline alone.

TABLE 4
Comparison of supernatants taken from precipitation
assay by absorbance A410.

| | |
|---|---|
| NAC-DPA | DPA |

Sample #

| | 1 | 2 | 3 | 1 | 2 | 3 |
|---|---|---|---|---|---|---|
| A410 | 0.587 | 0.59 | 0.629 | 0.388 | 0.396 | 0.401 |

### Example 7

Fluorescence of NAC-DPA complex at pH=5 was monitored as [DNA] was varied and [drug] was held constant (EX091205). Dipyridamole (DPA, m.w. 504.6) was prepared as 0.103 mM (0.0518 mg/ml) solution in water acidified with HCl. DNA was from HSDNA (428A) diluted 2 mg/ml in 25 mM KP pH 5 prev. heated to 80C. Diluted DNA in plate serially at 2, 1, 0.5, mg/ml etc. in 0.2 ml final. Added 0.05 ml, 0.1 ml, or 0.2 ml of DPA (0.0518 mg/ml) to DNA's and to 0.2 ml water only. Read fluorescence in black, NUNC plate, at EX485 nm/EM580 nm s=80 on Synergy HT1 plate reader.

### Example 8

Solubility Enhancement of Paclitaxel with NAC.

Materials: Paclitaxel 10.0 mg/ml (0.0118M, m.w. 853.92) in 80% ETOH. HSDNA (herring sperm degraded DNA 428A, Sigma-Aldrich) 10 mg/ml pH7 HSGDNA (genomic, Promega mw range 100 bp-3000 bp) 10 mg/ml. Multi-branched PEG-PEI2K was synthesized by reaction of pendant PEG propionic acid (Sun Bio Inc.) with 2000 mw branched, polyethylenimine catalyzed by 1-ethyl-3-(-3-dimethylaminopropyl) carbodiimide HCl (mw 191.7, EDC, TCI America Inc.) and/or NHS (N-hydroxysuccinimide). 1M potassium phosphate buffer pH5.

Aqueous paclitaxel solutions were prepared by combining 10 ul (0.01 ml) of paclitaxel stock (11.8 mM) with 2.0 ml H2O containing water only, HSGDNA, HSDNA, RNA, or HSDNA/pPEGPEI2K as indicated in the table. Samples were sonicated 3 min. with Branson Sonifier 450 @40% duty, at #3 output level. In 10 mM potassium phosphate pH5. The addition of KP caused the aqueous solution of paclitaxel to form a stable flocculate/precipitate which did not resolubilize and was stable against sonication and heating.

However, after sonication, DNA's formed stable, clear solutions with, preventing precipitation of paclitaxel. Let solutions stand 30 min, and read turbidity of 0.3 ml at A410 on the Synergy HT1. Samples were 10 min @2500 rpm on Beckman Allegra21R. The pellet was resuspended in 1.5 ml of 50% ETOH and OD230 nm was taken of a 0.3 ml sample in a Falcon UV clear plate on Synergy HT1. Prepared paclitaxel standard curve, 10 ul of 9.81 mM in 1 ml 50% ETOH was diluted 2×, serially in 5 vials at 0.5 ml final vol. The std curve was then used to calculate the mM paclitaxel from the pellet and the total percentage of solubilized paclitaxel per sample using %=100× (1-assy mM)/expected mM.

TABLE 5
Results of paclitaxel solubilization by NAC.

| | | | Sonicate | Turbidity |
|---|---|---|---|---|
| Sample | Desc. | DNA | @ pH 5 | OD410 |
| 1-3 | H2O | none | X | .122, .095, .134 |
| 4-5 | HGDNA | .1 ml | X | .104, .078 |
| 6 | HSDNA | .2 ml | X | 0.075 |
| 7 | HSDNA | .3 ml | X | 0.075 |
| 8-9 | HSDNA/pPEGPEI | .1 ml | X | .283, .252 |
| 10 | RNA | none | X | 0.093 |

### Example 9

Comparing toxicity of NAC-MTN with CD-MTN and MTN only (EX042105).

MCF7, COS7, 3T3, and BHK21 cells were plated out in duplicate rows of equivalent cell density in 100ul final volume. Initial cell densities were 2000 cells/well.

Preparing NAC-MTN, CD-MTN, and MTN only. Combined 180 ul of 0.05 mg/ml MTN with 750 ul 0.1 mg/ml herring sperm genomic DNA (HSGDNA, Promega), 1500 ul of 2× DMEM media containing 20% FBS to give 3.0 ml final volume and 12% final drug loading in the dsDNA. For MTN only, 180 ul of MTN was combined with 1320 ul H2O and 1500 ul 2× DMEM. The CD-MTN was prepared by combining 180 ul of MTN with 250 ul of 0.05 mg/ml betacyclodextrin-sulfylbutylether polymer BCD-SBE (K.Kosak, KKBiomed Inc), 1070 ul H2O, and 1500ul 2× DMEM to give 3 ml final and 3.6% drug loading in the CD carrier. The final [drug] at 1× was equivalent in all samples and was 6 uM MTN. After 48 hour incubation, 50 ul of either NAC-MTN, media only, CD-MTN, or MTN was added to the cells (to give 150 ul final) in the 96 well plate according to the following location scheme.

50 ul NAC-MTN added to column 3,7,11 rows A-D to give 2 uM final MTN.

50 ul of 1X DMEM media added to col 4,8,12 rows A-D to give media only control.

50 ul CD-MTN added to column 3,7,11 rows E-H to give 2 uM final MTN.

50 ul MTN added to column 4,8,12 rows E-H to give 2 uM final MTN.

BHK21 cells showed significant (est. 50%) toxicity after 20-24 hour incubation with MTN. Other cells MCF7, COS7 and 3T3 showed no toxic effect after 24 hours so incubation with drug was continued. Samples from col's 3,7,11; 4,8,12 rows A-H as above, were incubated for 24 hours then replaced with media.

After 24 hours, NAC-MTN showed higher protection against MTN toxicity or CD-MTN toxicity. All samples were replaced with 1× DMEM media as indicated. After an additional 24 hours, or 48 hours after adding samples to cells, the cells were assayed for viability by MTT assay.

Aspirated media in plate and replaced with 50 ul of 0.5 mg/ml MTT in 1× media, incubated 3 hours, aspirated, air dried 5 min and dissolved in 100 ul of isopropyl, read OD at A570.

TABLE 6
The % toxicity of NAC-MTN, CD-MTN, or MTN alone.
% tox. of formulations relative to media only

| Cell line | NAC-MTN | Media | CD-MTN | MTN |
|---|---|---|---|---|
| MCF7 | 15.66 | 0.00 | 50.20 | 48.39 |
| COS7 | 50.17 | 0.00 | 66.70 | 56.65 |
| 3T3 | 24.47 | 0.00 | 51.45 | 37.57 |
| BHK21 | 48.86 | 0.00 | 58.51 | 51.76 |

TABLE 7
The % difference in toxicity between NAC-MTN and CD-MTN or MTN.
NAC-MTN vs. MTN or CD-MTN as % tox difference

| Cell line | MTN | CD-MTN |
|---|---|---|
| MCF7 | 33 | 35 |
| COS7 | 6 | 17 |
| 3T3 | 13 | 27 |
| BHK21 | 3 | 10 |

Because of differences in metabolism and growth rate, each cell line produces a different MTT baseline signal. The baseline toxicity of cells treated with media only were used for computing % toxicities. After toxicity was normalized for each cell line, the relative differences in toxicities were compared. In MCF7's, NAC-MTN produced 33-35% reduction in toxicity compared with MTN only and CD-MTN. In COS-7's, NAC-MTN produced 17% reduction in toxicity compared with CD-MTN and 6% compared with MTN only. In 3T3's, NAC-MTN produced 27% reduction in toxicity compared with CD-MTN. In BHK21's, NAC-MTN produced 10% reduction in toxicity compared with CD-MTN but only 3% compared to MTN only.

When the data are viewed with respect to different cell lines, a large discrepancy in toxicity is observed between different cell lines treated with NAC-MTN. Comparison of TOX in MCF7 vs. COS7 or BHK21 show that MTN toxicity to fast growing cell lines is enhanced.

### Example 10

Comparing targeting of nucleic acid carriers composed of double stranded phosphodiester DNA and carriers composed of single stranded phosphorothioated DNA (EX051605).

Materials: HSGDNA, MTN, ODNAS1116 (phosphorothioated, single stranded DNA, and Beta-cyclodextrin polymer (BCD-SBE, KKBIOMED Inc.) were previously prepared in water at 10 mg/ml, 1 mg/ml, 0.95 mg/ml, and 10 mg/ml respectively.

Procedure: For NAC-MTN, combined 37 ul HSGDNA (herring genomic DNA, Promega Inc.), 1426 ul HBSS( Hank's buffered saline, Sigma-Aldrich), with 37 ul of MTN, followed by 1500 ul 2× DMEM media to give 3 ml final and 24 uM (24 micro-molar) MTN and 10% w/w drug loading of MTN. For NAC-MTN (AR1505) combined 62 ul of HSDNA (deg. herring sperm DNA, Sigma) with 2377 ul HBSS, 61.7 ul MTN, 2500 ul 2× media to give 5 ml final and 24 uM MTN and 10% w/w drug loading of MTN to HSDNA. For ODN-MTN, combined 200 ul of ODNAS1116, 531 ul HBSS, 19 ul MTN, and 750 ul 2× media to give 1500 ul final and 24 uM MTN or 12.3 ug/ml MTN and 10% w/w drug loading of MTN to ODN DNA. MTN only was prepared identically but without carrier to give 24 uM final MTN. Two hundred microliters (200 ul) of BCD-SBE polymer was combined with 1263 ul HBSS, 37 ul MTN, and 1500 ul 2× media to give 3 ml and 24 uM final MTN and 1.9% w/w drug loading of MTN. Media was DMEM supplemented with 10% FBS (HiClone labs.)

MCF7 (50K/well), COS7(50K/well), 3T3 (8K/well), and BHK2(8K/well) cells were seeded into two standard 96 well tissue culture plates and incubated 16 hours at 37C and 5% CO2.

Plate#1 Media was aspirated and cells treated with 100 ul of either NAC-MTN, CD-MTN, or MTN at 24 uM MTN final or 12.3 ug/ml MTN in all samples. Samples containing MTN were incubated 4 hours, removed, and fluorescence taken at EX620/EM680.

Plate#2 50 ul of sample NAC-MTN, CD-MTN, or MTN were added to plate containing 100 ul media to give 8 uM MTN final or 4.1 ug/ml MTN in all samples.

Samples were incubated 4 hours, removed, and MTN fluorescence taken at EX620/EM680. For uniformity, each sample was repeated in six wells, three in rows A-D and three in E-H and evenly spaced in every other column. Average MTN fluorescence was averaged from 6 wells. For each cell type, MTN fluorescence was highest in MTN treated cells, followed by CD-MTN, and lowest in NAC-MTN. To compare the uptake of MTN and MTN with carrier, and to normalize against differences in cell density, the fluorescence signal of NAC-MTN and CD-MTN treated cells was converted to a % of fluorescence signal of MTN treated cells.

Results. In plate#1 and plate#2, the MCF7 and 3T3 show consistently higher differences between uptake of NAC-MTN and MTN alone (see table). In BHK21 and COS7, the uptake of MTN alone is more equivalent to NAC-MTN. In plate#1 the differences in uptake between MTN alone and NAC-MTN in COS7 are less significant than in plate#2. Nucleic acid carriers comprising single stranded phosphorothioated oligonucleotide produced similar reduction in toxicity as did double stranded herring sperm DNA.

TABLE 8
The % reduction in fluorescence of CD-MTN,
HSDNA-MTN, ODN-MTN and HSGDNA-MTN, relative
to MTN alone (MTN = 0%)

| Plate#1 | CD-MTN | HSDNA-MTN(AR1505) |
|---|---|---|
| MCF7 | 34 | 36 |
| COS7 | 14 | 24 |
| 3T3 | 16 | 38 |
| BHK21 | 14 | 22 |

| Plate#2 | ODN-MTN | HSGDNA-MTN |
|---|---|---|
| MCF7 | 32 | 39 |
| COS7 | 28 | 32 |
| 3T3 | 35 | 37 |
| BHK21 | 12 | 19 |

### Example 11

The effect of plating time on the differential rates of uptake between NAC-MTN and MTN were assayed in cells treated <1 hour and 24 hours after plating.

TABLE 9
Showing volumes and other components required
for the assay (EX52505, example 12).

| | MTN | DNA 10 | | 2× | tot | drug % |
|---|---|---|---|---|---|---|
| Sample | 1 mg/ml | mg/ml | HBSS | media | vol | load |
| HSGDNA | 74.0 | 74.0 | 2852 | 3000 | 6000 | 10 |
| MTN only | 74 | 0 | 2926 | 3000 | 6000 | |
| media only | 0 | 0 | 1500 | 1500 | 3000 | |

On day 1, MCF7, COS7, 3T3, and BHK21 cells were plated out in a standard tissue culture plate. After incubation of 20 minutes samples were added to cells in rows A, C, E, and G. Cells were incubated at 37C in a standard atmosphere of 5%CO2.

On day 2, samples were added to rows D, F, and H and the fluorescence was obtained EX620/EM680 S=80. Samples were removed from A,C,E and G, replaced with media and fluorescence read again. On day 2 observed significant drug toxicity in cells of rows A,C, E, G. Sample addition was postponed in row B since MCF7 were not yet confluent.

On day 3, added samples to cells in row B containing confluent MCF7's, removed samples from rows D, F, and H replaced with media and read fluorescence.

The fluorescence background showed MTN fluorescence in cells prior to removing MTN.

Fluorescence data from cells taken after removal of media was used to quantitate total MTN retained in the cells. Fluorescence data were converted to mg/ml MTN using a standard curve prepared with free MTN or NAC-MTN. The MCF7 showed significantly lower MTN drug levels in established cells compared with the non-established cells (24 hour incubated group).

The percentage difference in fluorescence between cells treated with NAC-MTN or MTN alone was used to compare relative drug uptake. Surprisingly, NAC-MTN showed higher uptake in established BHK21 whereas the free MTN showed roughly equivalent uptake in both treatments.

Trends in toxicity correlated with uptake of MTN. In COS-7 the NAC-MTN toxicity was roughly equivalent in established and non-established cells. However, in MCF7's, toxicity was substantially different for established and non-established cells and reflect significant differences in fluorescence between NAC-MTN/MTN ratio in established (24hour) vs. non-established cells (<1 hour).

TABLE 10
Percentage difference of MTN fluorescence in cells
treated with either NAC-MTN or MTN alone.

| | Incb. | NAC-MTN | MTN | % Diff. |
|---|---|---|---|---|
| | time | FLUOR | FLUOR | FLUOR |
| MCF7 | <1 hour | 14583 | 16651 | 12 |
| MCF7 | 24 hour | 11118 | 18819 | 41 |
| COS7 | <1 hour | 10280 | 13814 | 26 |
| COS7 | 24 hour | 10810 | 15289 | 29 |
| NIH3T3 | <1 hour | 12587 | 18530 | 32 |
| BHK21 | <1 hour | 8097 | 9557 | 15 |
| BHK21 | 24 hour | 8987 | 10746 | 16 |

The MTN fluorescence ratios were obtained by dividing MTN fluorescence of cells incubated for 24 hours by fluorescence of cells incubated 30 minutes. The lower number indicates that a proportionately higher uptake occurred for cells treated after 24 hour incubation, which allowed adherence to the plate, than for cells treated 30 min after adding to the plate. The MCF7 showed the highest decrease in uptake of NAC-MTN after plate adherence followed by COS7 and BHK21. The uptake in BHK21 was roughly equivalent for NAC-MTN regardless of plate adherence, however it should be noted that BHK21 adhere very rapidly to the plate. The MCF7 cancer cells showed a disproportionately higher uptake as suspended cells or newly established cells than for established cells.

TABLE 11
MTN FLUOR Ratio of t(24)/t(<1)

| | NAC-MTN | +/−% | MTN | +/−% |
|---|---|---|---|---|
| MCF7 | 0.67 | 7 | 0.75 | 6 |
| COS7 | 0.85 | 1 | 0.70 | 2 |
| BHK21 | 0.96 | 3 | 0.73 | 1 |

Fluorescence data were converted to micromolar drug concentrations (uM MTN) using a standard curve for either nucleic acid bound or unbound drug, and drug uptake ratios were obtained by dividing the t(24 hour)/t(30 min.). The ratios of absolute drug concentration per well show more dramatic differences in uptake as shown below. The ratio of absolute drug concentration show that MCF7 cells have a significantly lower uptake of about 2-4 fold in established cells than COS7 or BHK21 respectively.

TABLE 12
Ratio uM MTN of t(24)/t(<1)

| | NAC-MTN | MTN | +/−% | +/−% |
|---|---|---|---|---|
| MCF7 | 0.5 | 1.2 | 22 | 9 |
| COS7 | 1.2 | 1.2 | 11 | 4 |
| BHK21 | 2.0 | 1.2 | 23 | 1 |

### Example 12

Comparison of toxicity of NAC-MTN in MCF7 and transformed primate kidney cells (COS7) with or without transfection agent and hydroxychloroquine. The experiment was designed to explore the differences in toxicity of nucleic acid-drug carriers delivered in two different cell types, MCF7 breast cancer cells and COS7 transformed primate cells. Different molar ratios of mitoxantrone (MTN) were complexed with genomic, double stranded DNA isolated from herring sperm (Promega) and incubated in cells for 72 hours. To test the effect of enhanced delivery, the NAC-MTN's were also formulated with a pendant PEG polyethylenimine(PEG-PEI2K, pPEG1020H, Kosak et al. U.S. application Ser. No. 11/305,688) and a lysosome disrupting agent, PEG-chloroquine at 50 uM. The chloroquine-PEG was previously prepared by conjugating chloroquine amine to polyethylene glycol (PEG-HQ, KKBiomed).

Prepared COS7 cells in plate at 40K cells/well. Incubated cells 24 hours and added 50 of NAC samples (EX021105). Assay for MTT activity. Aspirate media, replace with 50 ul of 0.5× media, read MTN fluorescence, then replace and add 50 ul 0.5 mg/ml MTT reagent incubate 4 hours, aspirate, let stand 2 min, add 100 ul 100% isopropyl and read A570. m All NAC-drug samples were stored one week at 4C and the experiment was repeated (EX021605) in MCF7 cells with identical conditions.

TABLE 13
Comparison of uptake and toxicity of NAC-MTN in MCF7
or COS7 at differing % drug loading and with or without
PEG-PEI (pPEG1020H) or PEG-hydroxychloroquine (PEG-HQ,
Kosak et al. US app. 11/305,688).

| | +/−% | 50 uM | | MTN |
|---|---|---|---|---|
| COS7 CELLS Sample | dev. | PEGHQ | % TOX | FLUOR. |
| NAC-MTN_9 | 3 | | 50 | 1067 |
| NAC-MTN_4.5 | 5 | | 46 | 670 |
| NAC-MTN_2.25 | 4 | | 43 | 421 |
| NAC-MTN_9 | 3 | PEGHQ | 54 | 1018 |
| NAC-MTN_4.5 | 7 | PEGHQ | 47 | 570 |
| NAC-MTN_2.25 | 8 | PEGHQ | 44 | 350 |
| NAC-MTN1020H_9 | 0 | | 63 | 1177 |
| NAC-MTN 1020H_4.5 | 4 | | 57 | 742 |
| NAC-MTN 1020H_2.25 | 3 | | 57 | 556 |
| | +/−% | 50 uM | | NORM |
| MCF7 CELLS Sample | dev. | PEGHQ | % TOX | FLUOR. |
| NAC-MTN_9 | 8 | | 19 | 2175 |
| NAC-MTN_4.5 | 6 | | 32 | 1449 |
| NAC-MTN_2.25 | 7 | | 34 | 739 |
| NAC-MTN_9 | 2 | PEGHQ | 24 | 2203 |
| NAC-MTN_4.5 | 1 | PEGHQ | 33 | 1241 |
| NAC-MTN_2.25 | 3 | PEGHQ | 36 | 619 |
| NAC-MTN 1020H_9 | 2 | | 21 | 2239 |
| NAC-MTN 1020H_4.5 | 1 | | 31 | 1347 |
| NAC-MTN 1020H_2.25 | 2 | | 31 | 791 |

After removal of the NAC-drug, fluorescence of the cells was taken. The MCF7 cells showed higher fluorescence than COS7. This was surprising given that the MCF7 showed significantly less toxicity than COS7.

### Example 13

Uptake study (EX051605) of NAC-drug prepared with 100% phosphorothioated DNA (PS-ODN) or genomic herring DNA (HSGDNA, Promega). MCF7, COS7, NIH3T3 (mouse fibroblast), and BHK21(hamster kidney cells) cells were plated out at 50K, 40K, 8K, and 8K per well into standard tissue culture plates. Prepared samples by combining MTN in HBSS (Hank's buffered saline) and 1× DMEM media with either phosphorothioated oligonucleotide (PS-ODN, HSGDNA, degraded HSDNA 40 bp avg, or cyclodextrin polymer to give 10% loading in DNA's. PLATE#1: Dispensed 100 ul sample into a sterilized plate using plate#1 format. Aspirated plate#1 and dispensed 100 ul sample into cell plate, pre-read fluorescence with samples (bkgd). Incubated 4 hours, removed to polyplate, replaced with media, read fluorescence. In plate#1, final MTN in cells was 24 uM or 12.3 ug/ml.

PLATE#2: Dispensed 50 ul from source plate into plate #2 to give 150 ul final vol 4.1 ug/ml, 8 uMfinal per well, pre-read fluorescence w samples (bkgd) incubated 5 hr, removed, replaced w/ media, read fluor.

MTT assay. Aspirated wells, add 0.05ml 0.5 mg/ml MTT in DMEM media incubated 4 hours, aspirated, air dried for 10 min. add 100 ul isopropyl w/0.1M HCl read at peak 406 nm.

In plate #2 add 100 ul 100 isopropyl read peak at A570.

TABLE 14
% Difference in fluorescence of NAC-MTN normalized
to MTN fluorescence in MCF7, 3T3, COS7, and BHK21.

| | MTN only | NAC-MTN HSDNA |
|---|---|---|
| MCF7 | 0 | 36 |
| COS7 | 0 | 24 |
| NIH3T3 | 0 | 38 |
| BHK21 | 0 | 22 |

TABLE 15
% Difference in fluorescence of NAC-MTN (prepared
with PS-ODN or genomic DNA) normalized to MTN fluorescence
in MCF7, 3T3, COS7, and BHK21.

| | ODN-MTN | MTN only | NAC-MTN HSGDNA |
|---|---|---|---|
| MCF7 | 32 | 0 | 39 |
| COS7 | 28 | 0 | 32 |
| NIH3T3 | 35 | 0 | 37 |
| BHK21 | 12 | 0 | 19 |

The MTN fluorescence data show that uptake of NAC-MTN is higher in rapidly growing cell lines. The percentage difference in fluorescence was taken as the difference in fluorescence between the cells treated with drug only and the NAC-drug carrier. Smaller % differences indicate that uptake of NAC-MTN and MTN was more equivalent. Cell lines with the highest proliferation rates such as BHK21 and COS7 showed the most dramatic uptake rates and the lowest % difference between NAC-MTN and MTN only. However, in the slower proliferating cells MCF7 and NIH3T3, the differences in fluorescence were significantly higher, since uptake of NAC-MTN is lower in these cells relative to MTN alone. Differences were observed between NAC-MTN composed of the PS-ODN and the much larger genomic DNA. NAC-MTN composed of PS-ODN's showed slightly higher uptake rates in faster dividing cell lines than NAC-MTN composed of HSGDNA.

### Example 14

Preparation of NAC-DOX with phosphodiester DNA or phosphorothioated DNA and formation of PEG shielded micelles (EX112304).

Combined 0.063 m (0.949 mg/ml) 100% phosphorothioated ODN (PS-ODN) with 0.125 ml H2O, 6 ul of 0.041 mM doxorubicin HCl and 0.015 ul of 2.65 mg/ml pPEG propionic acid-hydrazine (pPEG914C). PPEG914C was previously prepared by reaction of hydrazine with pendant PEG propionic acid catalyzed by EDC [1ethyl-3-(3-dimethylaminopropyl) carbodiimide HCl] and n-hydroxy succinimide. The ODN-PEG micelles were added to MCF7 cells 6000/well to give 1.2 uM (micromolar) DOX and incubated for 96 hours. Cell viability was assayed by MTT assay.

HSDNA or HSGDNA were combined with DOX to give 8 uM stock in water. HSDNA-DOX or HSGDNA-DOX was then combined with pPEG1020H or pPEG914C to form PEG shielded micelles with a PEG outer surface and DNA-DOX core at 0.015 or 0.016 PEG/phosphate molar ratio.

EX021105 HSDNA-MTN complex delivered with pPEG-Protamine (pPEG11105C, Kosak et al. U.S. application Ser. No. 11/305,688). HSDNA-MTN complex were prepared at 9, 4.5, and 2 uM final MTN and combined with pPEG 11105C at 80:1 protamine:DNA ratio. The particles were incubated with cells and toxicity assayed by MTT. The percentage toxicity was 86%, 83%, and 80% respectively for 9 uM, 4.5 uM, and 2 uM final MTN. Post incubation, MTN fluorescence was taken at EX620/EM680 and was 768, 585, and 457, respectively.

TABLE 16
PEG NAC-DOX micelle 914C @1.2 uM DOX

| | % TOX | | |
|---|---|---|---|
| | 23 | 17 | 0 |
| [PS-DNA] | 0.149 | 0.074 | 0.037 |

TABLE 17

| | pPEG- | .207 mM | | | Tot. |
|---|---|---|---|---|---|
| Sample prep. vol. in ul | cpd. | DOX | DNA | H2O | vol. |
| (1) HSDNA428_DOX_8uM | | 40 | 400 1 mg/ml | 560 | 1000 |
| (2) HSGDNA_DOX_8uM | | 40 | 40 10 mg/ml | 920 | 1000 |
| HSDNA428_DOX_8uM_pPEG1020H | 13 | | 200 (1) | 0 | 200 |
| HSGDNA_DOX_8uM_pPEG1020H | 13 | | 200 (2) | 0 | 200 |
| HSDNA428_DOX_8uM_pPEG914C | 15 | | 200 (1) | 0 | 200 |
| HSGDNA_DOX_8uM_914C | 15 | | 200 (2) | 0 | 200 |

### Example 15

In vivo comparison of NAC-MTN and MTN in mice after single dose (EX102005). Mice n=2, were injected i.v. with equivalent amounts of a MTN drug formulated as a single dose of either NAC-MTN or MTN alone.

Procedure: Prior to formulation of MTN with carrier, the DNA carrier was standardized and assayed for loading limits with MTN by absorbance spectrometry. Four different DNA carriers preparations were compared for relative loading with MTN (EX101705). MTN (mitoxantrone HCl, Sigma-Aldrich) was prepared by diluting 81.8 mg (Sigma-Aldrich) in 8.16 ml sterile water to give 10 mg/mI stock and 19.3 mM final. A 0.4 ml volume of each NAC-MTN or MTN formulation was prepared for injection in HBSS (Hanks Buffered Saline, Sigma-Aldrich) as indicated.

The NAC-MTN injection was prepared by combining fifteen microliters (15 ul) 10 mg/ml MTN in 325 ul of HBSS (Hank's buffered saline, Sigma-Aldrich) solution. Herring sperm DNA HSDNA (50 mg/ml stock neutralized 0.2 um filtered) was then added to give a turquoise blue color. For MTN only injection, fifteen microliters (15 ul) 10 mg/ml MTN was diluted in 385 ul of HBSS solution. White mice, n=2, 24 g each, were then tail vein injected with two hundred microliters (200 ul) of either NAC-MTN or MTN to give 0.075 mg MTN per mouse. After 1 hour, the mice were sacrificed and the liver, spleen, kidneys, lungs, and heart removed. Liver and kidneys were treated with 1 ml or 0.5 ml respectively of 1% sodium dodecyl sulfate (SDS) and macerated with a teflon pestle to yield 2 ml of liver homogenate and 1 ml of kidney homogenate. One milliliter (1 ml) of each homogenate was then transferred to a 1.7 ml centrifuge tube and centrifuged for 10 min at 10000 rcf. Supernatants were removed and fluorescence of 0.3 ml of samples was read at 620/680 nm s=50. Significantly higher MTN fluorescence was observed in liver and kidney of MTN treated mouse than in NAC-MTN treated mouse. Total MTN was calculated by converting fluorescence data to mg/ml MTN using a MTN standard curve. The mg/ml MTN was then multiplied by total vol. homogenate to obtain total MTN per organ. The density of MTN in tissues was found to be significantly higher in the heart than in other tissues.

TABLE 18
MTN distribution in major organs of the mouse expressed as MTN ug/g
tissue.
Density of MTN in tissues of each organ (ug/g)

Sample

| | Liver | Kidney | Heart | Lung |
|---|---|---|---|---|
| NAC-MTN | 5 | 21 | 76 | 34 |
| MTN | 10 | 49 | 96 | 43 |
| Ratio | 1.9 | 2.3 | 1.3 | 1.3 |

### Example 16

In vivo comparison of NAC-MTN and MTN in mice after multiple dosing (EX102205). Mice n=2, were injected i.v. with repeated dose of either NAC-MTN or MTN alone. MTN administered was the same for each mouse.

MTN was prepared by diluting 81.8 mg (Sigma-Aldrich) in 8.16 ml sterile water to give 10 mg/ml stock and 19.3 mM final. A 0.4 ml volume of each NAC-MTN or MTN formulation was prepared for injection in HBSS or H2O (Hanks Buffered Saline, Sigma-Aldrich) as indicated below. For NAC-MTN injection, 45 ul 10 mg/ml MTN was diluted first in 325 ul of HBSS solution, then 180 ul of herring sperm DNA (HSDNA, 50 mg/ml stock neutralized, 0.2 um filtered) was added to give a turquoise blue color. For MTN formulation, water was found to be more stable against precipitation of MTN. In MTN injection, 45 ul 10 mg/ml MTN was diluted in 385 ul of H2O.

On day 1, white mice, n=2, 24 g each, were tail vein injected by a micro-catheter with 0.16 ml at 14:50 or 0.15 ml at 15:30, respectively of either NAC-MTN or MTN. A second injection was followed at 17:00 and 16:50 of 0.2 ml each of NAC-MTN or MTN. To quantify total MTN retained, mouse urine was collected after 1st and 2nd injection. Total MTN was assayed in mouse urine immediately after 1st injection and second injection by reading MTN fluorescence 620/680 s=50 of a 0.2 ml sample in a styrene well, bottom read. Data showed unexpectedly more retention of MTN in the MTN treated mouse compared with NAC-MTN treated mouse. A third dose of 0.2 ml was given the next morning, 22 hours after 1st injection (day 2), of either NAC-MTN or MTN. Neither mouse showed any toxic effects. Twenty four hours after first injection (day 2), mice were sacrificed and MTN assayed in major organs. The liver, kidneys, heart, and lung were homogenized at high speed 50,000 rpm, and treated with 1% SDS (sodium dodecyl sulfate). The homogenates were frozen twice and centrifuged for 30 minutes at 14000 rcf.

TABLE 19
Assay results of MTN distribution in major organs reported
as MTN mg/g tissue. The ratio shows MTN accumulation
is more than 2 fold greater in heart and lung, and almost
3 fold greater in liver, in mice treated with MTN alone
than in mice treated with NAC-MTN.

| Sample | Liver | Kidney | Heart | Lung |
|---|---|---|---|---|
| NAC-MTN | 0.028 | 0.267 | 0.015 | 0.011 |
| MTN only | 0.077 | 0.261 | 0.033 | 0.027 |
| Ratio | 2.8 | .98 | 2.2 | 2.4 |

TABLE 20
Total MTN recovered from either NAC-MTN or
MTN treated mouse per time after injection.

| | MTN | | MTN | MTN | Collect | MTN |
|---|---|---|---|---|---|---|
| | dose | Inject. | dose | Recov. | Time | % dose |
| Formulation | vol | time | tot. mg | mg | (hr) | recov. |
| NAC-MTN | 0.16 | 0 | 0.18 | 0.0138 | 0-2 | 7.7 |
| MTN | 0.15 | 0 | 0.17 | 0.0049 | 0-2 | 2.9 |
| NAC-MTN | 0.2 | 3 | 0.225 | 0.0429 | 3-12 | 19 |
| MTN | 0.2 | 3 | 0.225 | 0.0279 | 3-12 | 12 |
| NAC-MTN | 0.2 | 22 | 0.225 | 0.0369 | 12-24 | 16 |
| MTN | 0.2 | 22 | 0.225 | 0.0297 | 12-24 | 13 |

### Example 17

In-vivo study of NAC-MTN and MTN distribution in mice and confirmation of stable NAC-MTN complex in blood stream.

Procedure. NAC-MTN was prepared at 5% drug loading with 1.125 mg MTN/ml and 22.5 mg DNA/ml. MTN was prepared in saline at 1.125 mg/ml MTN final. White mice, n=2 were tail vein injected with 0.25 ml of either NAC-MTN or MTN alone. Total MTN dosage per mouse was 0.281 mg. Following injection, NAC-MTN (nucleic acid bound MTN) or free MTN was detected in mouse urine within two hours post injection and as late as 8 hours. Post treatment, neither mouse showed any toxic effects. After 8 hours, mice were sacrificed, the liver, kidney, spleen, heart, and lung removed, homogenized at high speed (50,000 rpm), and treated with 1% SDS (sodium dodecyl sulfate). The homogenates were frozen twice and centrifuged for 30 minutes at 14000 rcf. MTN fluorescence was detected in supernatants at excitation (620 nm)/emission(680 nm) s=80 in 0.3 ml sample (Synergy HT1 Biotek). MTN was quantified using an MTN standard curve. MTN was quantified in mouse urine by absorbance of 0.2 ml sample at 665 nm, and converted to mg/ml using an MTN standard curve.

TABLE 21
MTN recovered in mouse urine at various time
points and expressed as % of total dose.

| Group | Formulation | FLUOR. | Elapsed Time (hr) | MTN % recov. |
|---|---|---|---|---|
| 1 | NAC-MTN | 14595 | 2.5 | 8.4 |
| 2 | MTN | 4086 | 2.5 | 1.9 |
| 1 | NAC-MTN | 5734 | 5 | 2.0 |
| 2 | MTN | 4405 | 5 | 1.6 |
| 1 | NAC-MTN | 6294 | 8 | 2.1 |
| 2 | MTN | 4455 | 8 | 3.8 |

Results. The NAC-MTN was cleared more rapidly from the blood than MTN alone. At 2.5 hours post-injection, MTN clearance from the blood was about 4 times higher in NAC-MTN treated mouse than in MTN treated mouse. To determine if NAC-MTN was excreted in urine as a complex, MTN fluorescence due to unbound NAC was assayed by an addition method. DNA was added to urine from both groups and subsequent change in fluorescence was measured. Change in fluorescence was significant for MTN treated group, due to the binding of DNA with the MTN. However, change in fluorescence was negligible in NAC-MTN group, since MTN is already complexed with NAC. From these data, the NAC-MTN complex is shown to be highly stable and surprisingly intact, after circulation and subsequent elimination from the mouse.

TABLE 22
The relative amount of NAC complexed MTN or
unbound MTN, in mouse urine, as % change in
fluorescence by DNA addition method.

| Group | Formulation | Elapsed Time (hr) | % Change Fluor. |
|---|---|---|---|
| 1 | NAC-MTN | 2.5 | −1 |
| 2 | MTN | 2.5 | 83 |
| 1 | NAC-MTN | 5 | 12 |
| 2 | MTN | 5 | 66 |
| 1 | NAC-MTN | 8 | 21 |
| 2 | MTN | 8 | 71 |

### Example 18

Preparation of NAC-drug compositions from DOX-pPEG, and trioxsalen(TX)-pPEG conjugates (EX102004).

Materials: HSDNA or HSGDNA 1 mg/ml, glutaraldehyde 10 mM, doxorubicin HCl 379.99 g/mol (Chemwerth Inc. 1.2 mg/ml in H2O), trioxsalen amine (TX) mw 293 g/mol, 2.5 mg/ml in DMF, Schiff's reagent 1× (Sigma-Aldrich), pPEG-Ald914A#2 24.7 mg/ml, pPEG-Hdz914B 37.2 mg/ml. Pendant PEG(15) propionic acid was obtained from SunBio Inc.

Procedure: HSDNA (herring sperm DNA) and HSGDNA (genomic) in 1020A, C, and E, were prepared with trioxsalen amine (TX), or doxorubicin HCl, purified by dialysis in 2K (2000 cut-off) spectropore membrane, reacted with glutaraldehyde for 3 hours at 45C, and further purified by dialysis at 2K, 16 hours. Volumes and concentrations for the reactions are given in the following table. To calculate [drug] or [aldehyde], absorbance A570 was taken of each compound alone or after reaction with Schiff's reagent, and converted to mg/ml with a standard curve.

At 10% drug loading, the free doxorubicin in NAC-DOX was less than or equal to 4%. Post-dialysis of NAC-DOX, total doxorubicin recovered was 96%, indicating only 4% loss of drug.

Pendant PEG propionic acid (PPEG) was previously derivatized with hydrazine by catalysis with 1-ethyl-3-(-3-dimethylaminopropyl) carbodiimide HCl (mw 191.7, EDC, TCl America Inc.) and/or NHS (N-hydroxysuccinimide) to form pPEG-Hdz. PPEG-Hdz-Aldehyde was previously prepared by reacting glutaraldehyde (Glut.) with pPEG-Hdz. After characterization of 1020A-F products, the DNA-TX-amine or DNA-DOX was reacted with pPEG-aldehyde or pPEG-Hdz for 4 hours at 45C. The NAC-drug-pPEG conjugates were then dried, reconstituted in water, and assayed for activity in cell culture by the MTT assay.

TABLE 23
Intermediate compounds prepared in synthesis of NAC-drug-pPEG conjugates.

| | | DNA | DOX or | 10 mM | DOX | | % |
|---|---|---|---|---|---|---|---|
| | | 1 mg/ml | TX | glut. | or TX | | entrap |
| Sample | description | ml | ml | ml | % load | A570 | DOX |
| 1020A | HSDNATX | 1 | 0.04 | none | 10 | 0.0435 | |
| 1020B | HSDNATXAld | 6 | 0.24 | 2 | 10 | 0.136 | |
| 1020C | HSGDNATX | 1 | 0.04 | none | 10 | 0.012 | |
| 1020D | HSGDNATXAld | 6 | 0.24 | 2 | 10 | 0.0775 | |
| 1020E | HSDNADOX | 1 | 0.08 | none | 9.6 | 0.0465 | 96% |
| 1020F | HSDNADOXAld | 2 | 0.16 | 0.68 | 9.6 | 0.083 | 96% |

### Example 19

Preparation of poly(ethylene glycol)-DNA conjugate.

Synthesis of pPEG-NHS (pendant PEG N-hydroxy succinimide ester). Dissolved 0.25 g pPEG (0.0125 mmol) in 1 ml H2O to produce a clear solution. Added 15× mol ratio of EDC, 71.6 mg and after 10 min. formed gel. Added approx. 2 ml H2O to the reaction caused the polymeric gel to dissolve. After 20 min. 120 mg of N-hydroxy succinimide (115 mg/mmol) or 5.3 mol ratio was added, and the rxn was allowed to stand @25C for 2 hours. pPEG-NHS was standardized with ethyl carbazate. The pPEG-NHS product was diluted serially in H2O at 0.25×, 0.125× etc to 0.1 ml final volume. Added 0.05 ml 1 mM ethyl carbazate to all wells and allowed reaction for 30 min at 25C. followed by addition 0.1 ml 0.1M Na3PO4 and 0.05 ml 0.125% TNBS (picryl sulfonic acid). Let stand 10 min then read A515. Approx. first 4 wells showed the absence of primary amine groups, indicating that ethyl carbazate was consumed in rxn with pPEG-NHS. The pPEG-NHS products were purified by G25 sephadex column. Loaded entire product volume onto column (2 ml) and pre-elute 3 ml. Collected 25 drops/well/0.75 ml, eluent=H2O. Absorbance at 230 nm and 290 nm produced a single peak.

Phosphorothioated, single stranded oligodeoxynucleotides ODN's were previously synthesized with a 5′ amine overhang and HPLC purified (R. Shackman, University of Utah). PEG-DNA conjugates were prepared by combining 50 ul of 0.801 mg/ml ODN 5′amine (with 8 ul of 10 mg/ml pPEGNHS 1104B.

### Example 20

Synthesis of AZT-DAA drug conjugate with ester linkage. Synthesis of diaminoacridine (DM)-succinate. In 10 ml anhydrous pyridine, combined 24.5 mg of 3,6 diaminoacridine (0.1 mmol, Sigma-Aldrich) with 0.5 g of succinic anhydride and 0.125 g DMAP (1 mmol, Fluka Inc.). After purification of the DAA-succinate, the DAA-succinate is reacted with AZT 3′-azido-3′-deoxythymidine (Sigma-Aldrich) in a modified procedure of Gunaseelan et al. Bioconjugate Chem 15:1322-1333(2004), under anhydrous conditions in the presence of carbodiimide catalyst, 1-ethyl-3-(3-dimethylaminopropyl) carbodiimide hydrochloride, and DMAP (dimethylaminopyridine) to yield AZT-DM conjugate.

### Example 21

Preparation of NAC-drug from nucleoside and nucleotide-drug complex. Prepared azidothymidine (AZT) at 2.7, 1.35, 0.625, mg/ml etc. in 0.1 ml final water. Added 20 ul of 0.1 mg/ml DAPI followed by 50 ul of Hank's buffered saline (Sigma-Aldrich) and read fluorescence EX360/460 s=50. Prepared three dilution curves of dNTPs (25 mM each dATP, dCTP, dGTP, dTTP) at 100, 50, 25, 12.5 mM etc in 50 ul H2O final. Added 20 ul of 0.1 mg/ml MTN, DOX, or DAPI per well, followed by 30 ul of 1XHBSS to give 100 ul final and read fluorescence EX360/460. Fluorescence enhancement was more significant in dNTP-DAPI than AZT-DAPI. MTN-dNTP and DAPI-dNTP at high N/P ratio were found to produce turbid solutions and measured by A410 nm (data not shown). AZT-DAPI was less stable as a complex than dNTP-DAPI in the presence of potassium phosphate than dNTP-DAPI. DNTP-DOX resulted in significant fluorescence quenching at high N/P ratio.

### Example 22

Complexation of deoxynucleoside with HSDNA by inclusion of 4′, 6 diamidino-2-phenylindole (DAPI). In 10 ul of H2O, prepared 10, 5, 2.5, and 1.25 ul of 10 mg/ml HSDNA (428A) in four wells, leaving the fifth as blank/water only. Added 20 ul H2O, 10 ul of 0.1 mg/ml DAPI, and 10 ul of 5% ficoll to all wells and read fluorescence EX360/460. Then added 10 ul of 100 mM dNTPs to all wells and read fluorescence again.

TABLE 23
Preparation of HSDNA-DAPI-dNTP complex and results

| | ug DNA | | | | |
|---|---|---|---|---|---|
| | 100 | 50 | 25 | 12.5 | 0 |
| ug DAPI | 1 | 1 | 1 | 1 | 1 |
| ug dNTP | 330 | 330 | 330 | 330 | 330 |
| Fluor. w/o dNTP | 27021 | 18160 | 12202 | 9021 | 12090 |
| Fluor. w dNTP | 36283 | 26969 | 18742 | 14486 | 11450 |
| difference in F | 9262 | 8809 | 6540 | 5465 | −640 |

### Example 23

Gel migration of DNA-DAPI-nucleoside complex.

Protocol. In col. 1 combined 10 ul DAPI, dNTP, HSDNA and ficoll, col. 2 combined 10 ul DAPI, 20 ul dNTP, 10 ul ficoll col. 3 combined 10 ul DAPI, HSDNA, 20 ul ficoll, and col. 4 combined 10 ul DAPI, 30 ul dNTP, 10 ul ficoll. Agarose gel was prepared at 1.3% (FMC inc.) in tris-borate EDTA buffer. Loaded 10 ul of each DAPI-dNTP-HSDNA, DAPI-dNTP, DAPI-HSDNA, or DAPI-dNTP(3×) on the gel. Results. DNTP-DAPI-HSDNA produced a concise but widened, blue fluorescent band that migrated toward the positive pole. HSDNA-DAPI band was more narrow. DAPI-dNTP or free DAPI bands migrated towards the negative pole.

### Example 24

Preparation of NAC-drug with drug binding ligand (aptamer). Procedure: the model peptide drug, auristatin E (AE) is synthesized according to a procedure described by Pettit et al, U.S. Pat. Nos. 5,635,483, and 5,780,588 incorporated herein by reference. A DNA template library is synthesized with a solid-state DNA synthesizer (Applied Biosystems, CA) to contain a randomized library sequence of 40 nucleotides, flanked by two primer template sequences at the 5′ and 3′ ends [Lupold et al, Cancer Res. 62:4029-4033 (2002), J. Pollard “Nuc. Acid Pools” (1998)]. AE is conjugated to a M450 magnetic bead particle (Dynal Biotech). AE-M450 beads are incubated with the template library, the desired AE-template complex is purified by magnetic bead separation, and the nucleic acid template is amplified by polymerase chain reaction (PCR) with appropriate PCR primers, 1.25 units Taq polymerase, dNTP's, 2 mM MgCl, and 1× PCR buffer for 35 cycles. The AE binding sequence is determined by sequencing the PCR product (Myriad Genetics, UT). The AE binding sequence is synthesized by conventional phosphoramidite chemistry, de-salted, and HPLC purified (DNA synthesizer, Applied Biosystems, CA). Optimal nucleic acid-AE drug loading is determined with fluorescence assay by exclusion of DAPI or diaminoacridine. NAC-AE complex are then evaluated for drug masking, differential uptake, and co-transport of NAC-AE, in-vitro, at varying nucleic acid:peptide w/w ratio, for example, at between 0.1% and 90% w/w and diluted to between one nanomolar (1 nM) and 100 nM effective [peptide] in cell culture with MCF7 human breast cancer cells, and toxicity assayed by MTT assay. The NAC-auristatin complex can then be formulated in a suitable vehicle for administration in a cancer treatment such that the percentage bound drug ranges between 0.1% and 100% (w/w), but more preferably between 1% and 100% (w/w).

### Example 25

Preparation of bi-functional, NAC-auristatin complex, comprising an auristatin binding sequence and a sequence that binds a cell surface protein specific to prostate cancer. Nucleic acids that contain an auristatin binding sequence and optionally a targeting sequence, such as prostate specific membrane antigen (PSMA), PSMA sequence disclosed by Lupold et al, Cancer Res. 62:4029-4033 (2002)] are synthesized by phosphoramidite chemistry, de-salted, and HPLC purified. Optionally, the nucleic acids are each synthesized with a terminal amino group and coupled via a 20K bifunctional PEG with N-hydroxy succinimide (PEG-NHS ester) at pH 7 for 2 hours at 25C. The bifunctional NAC or bifunctional NAC-PEG conjugate is combined with auristatin E to form the NAC-auristatin complex, such that the % bound drug in the composition ranges between 0.1% and 100% drug (w/w) and the molar ratio % of free PSMA ligand that is available to bind cell surface antigen are between 100% and 0.01%, but more preferably between 100% and 10% (mol/mol).

### Example 26

Drug toxicity of MTN, DOX, and DAPI after complexing with deoxynucleotides or azidothymidine (EX021206). All dNTP-MTN, dNTP-DOX, and dNTP-DAPI were brought up with H2O and heated at 75C for 10 minutes. Note that dNTP-MTN produced fine particulates in wells 8-11 only, which settled to the bottom of the styrene well, forming a coating of microscopic droplets. The dNTP-MTN particles caused precipitation of most of the MTN from solution. However, unlike previous precipitates of MTN, these particles were completely solubilized by heating. Diluted dNTP-MTN or dNTP-DOX to 200 ul final to give 1× DMEM (DMEM w 10% FBS) and 0.1 mg/ml final, 19.3 uM MTN or 17.2 uM DOX and 1.6, 0.8, 0.4 etc. mg/ml dNTP final. Heated dNTP-MTN or dNTP-DOX particles to 70C cool for 1 min, added 100 ul 2× DMEM and dispensed 50 ul to MCF7 cells (20K/well) to give 250 ul total, 3.9 uM final MTN or 3.4 uM DOX in all cells, col 2-7 row B,C and D,E.

AZT-DAPI. Two hundred microliters (200 ul) of 10 mM azidothymidine (AZT) was heated at 75C for 10 minutes. 100 ul of the 10 mM AZT was combined with 100 ul of 2× DMEM media to give 200 ul final. Combined 100 ul of 10 mM AZT with 100 ul H2O in col#1 to give 5 mM, and diluted serially ½ thereafter in col. 2, 3.. 11 to give 2.5, 1.25 mM etc in 100 ul final. Col. 12 left as DAPI only control. Added 20 ul of 0.1 mg/ml DAPI to each of 12 wells, containing 2.5, 1.25 mM.. etc. AZT. Heated solution to 75C for 10 min and added 100 ul of 2× DMEM media to give 200 ul final. Repeated for AZT only, diluting to 5, 2.5, 1.25 mM etc in col 1-11, to 100 ul final vol and added 100 ul 2× DMEM to give 200 ul final. Added 20 ul of AZT-DAPI or AZT to cells containing 200 ul media. Final AZT was 0.5 mM, 0.25 mM, 0.125 mM etc in cells for AZT-DAPI or AZT alone. Blue fluorescence of AZT-DAPI was observed to shift in the first two dilutions at EX308 nm. Prepared dNTP-DAPI in 100 ul of 1× DMEM by drying, rehydrating, and heating 80C in 50 ul H2O followed by add'n of 50 ul 2×DMEM. 50ul of dNTP-DAPI was then vertically in plate to B9-E9, B10-E10, and B11-E11 to give 0.004 mg/ml DAPI (11.4 uM) in cells giving 3.3, 1.65, 0.825 mg/ml etc or 10, 5, 2.5 mM dNTP etc. in cells. Cells were incubated 2 days, followed by MTT assay. DNTP's showed a 30% reduction in DOX toxicity compared with DOX alone, even at high drug loading (w/w >60%), whereas MTN showed almost no reduction after drug load exceeded 1% ( w/w). Surprisingly, dNTP and dNTP-DAPI at 1.65mg/ml final and 0.1 % drug/dNTP (w/w) produced negligible toxicity while dNTP-DAPI at 3.3 mg/ml and 0.1% drug/dNTP (w/w) showed significant toxicity.

### Example 27

Preparation of ODN-cholesterol-DOX, ODN-oleic acid-DOX, ODN-folic acid-DOX, and ODN-mAb-DOX. Phosphodiester oligonucleotides (ODN) are synthesized with a 5′ amino overhang (Synthegen Inc.). The amino labeled ODN's are suitably reacted with 2:1 mole ratio of either cholesterol chloroformate (Sigma-Aldrich), NHS-Oleate (379.5 g/mol, Sigma-Aldrich), or NHS-folic acid (dissolve 1 g folic acid in 50 ml DMSO, 1 ml triethylamine, 0.5 g N-hydroxysuccinimide and 0.5 g dicyclohexylcarbodiimide [Steenis et al. J. Cont. Rel. 87:168 2003] to form ODN-cholesterol, ODN-oleic acid, or ODN-folic acid conjugates. The ODN conjugates are combined with doxorubicin HCl at 5% DOX:ODN w/w % to form NAC-DOX complex and formulated in suitable aqueous vehicle for parenteral administration. Briefly, the ODN-mAb is prepared by combining the ODN-5′-NH2 with succinic anhydridein anhydrous solvent at 1:2 mole ratio, reacting overnight at 25C, purifying the ODN-COOH product by dialysis with 2K spectropore membrane, and reacting the ODN-COOH product with the antibody (Ab) in the presence of dicyclohexylcarbodiimide or N-cyclohexyl-N′-[2′-morpholinoethyl]-carbodiimide. ODN-mAb#2 is prepared by modification of a procedure disclosed in U.S. Pat. No. 4,699,784, wherein the carbohydrate portion of the mAb is oxidized with sodium metaperiodate to form reactive aldehydes, the mAb-aldehydes are reacted with ODN-5′-NH2, and the Schiffs base intermediate is reduced with 10× mole ratio of cyanoborohydride. The Ab is preferably selected from a chimeric humanized or primatized antibody (Vector Labs Inc.). Doxorubicin is combined with the ODN-Ab to form ODN-Ab-DOX complex at 5% w/w.

### Example 28

DNA-DOX-Ab complex. DNA-DOX complex are prepared at 10% w/w. Anti-nucleic acid antibody, anti-BrdU Ab (Sigma-Aldrich, BU-33) is combined with the DNA-DOX complex at 1:1 mol ratio to form the DNA-DOX-Ab complex and administered to tumor bearing mice by tail vein injection.

### Example 29

Synthesis of paclitaxel-DAA drug conjugate with ester linkage. Synthesis of paclitaxel-succinate. In 10 ml anhydrous pyridine, combine 0.1mmol of hydroxypaclitaxel (Sigma-Aldrich) with 0.5 g of succinic anhydride and 0.125 g DMAP (1 mmol, Fluka Inc.). After overnight reaction and purification of the paclitaxel-succinate, the paclitaxel-succinate is reacted with 2:1 mol ratio of DAA (Sigma-Aldrich) for 16 hours under anhydrous conditions in the presence of carbodiimide catalyst, 1-ethyl-3-(3-dimethylaminopropyl) carbodiimide hydrochloride, and DMAP (dimethylaminopyridine) to yield paclitaxel-DAA conjugate (Tax-DM). Tax-DM is then combined with HSDNA to form a HSDNA-Tax-DAA complex and administered to tumor bearing mice.

TABLE 24
Examples of various NAC-drug compositions

| Formulation | NAC | Drug (s) | Target |
|---|---|---|---|
| NAC-DOX | DNA | doxorubicin | cancer |
| NAC-MTN | DNA | mitoxantrone | cancer |
| NAC-TAX | aptamer | paclitaxel | cancer |
| NAC-(TAX-DAA) | DNA | paclitaxel-DAA | cancer |
| NAC-AE | aptamer | peptide | cancer |
| PEG-NAC-interferon | aptamer-PEG | β-interferon | hepatitis |
| NAC-antibiotic | aptamer | NAC-tetracycline | bacteria |
| NAC-AZT | DNA-NABS | azidothymidine | AIDS |
| NAC-VIR | DNA | Viramidine(+) | hepatitis |
| NAC-MTN | nucleotide | AZT, mitoxantrone | AIDS |
| NAC-CAL | DNA | calicheamicin-DAA | cancer |

What is claimed is:

1. A nucleic acid carrier-drug composition, suitable for delivering a drug to a cell, comprising a complex formed between a nucleic acid carrier and a drug, wherein the drug is non-covalently associated with the nucleic acid carrier.

2. The composition of claim 1, wherein the drug is selected from doxorubicin.

3. The composition of claim 1, wherein the drug is selected from mitoxantrone.

4. The composition of claim 1, wherein the drug is selected from a nucleic acid intercalator drug, a groove binding drug, carboplatin, cisplatin, platinates, taxanes, paclitaxel, tubulin binders, methotrexate, calcheamicin, camptothecin, cylosporine, DNA alkylator, anti-mitotic, anti-neoplastic, alkyloid, antibiotic, anti-viral, anti-inflammatory, protein drug, peptide drug, folate antagonist, nucleoside drug, opioid, neorologicals, steroid, nucleotide drug, anti-angiogenesis, enzyme inhibitor, or a protease inhibitor.

5. The composition of claim 1 wherein the drug is a drug conjugate.

6. The composition of claim 1, further comprising a nucleic acid binding spacer (NABS).

7. The composition of claim 7, wherein the NABS is selected from a nucleic acid intercalator, groove binder, protein, polypeptide, oligopeptide, or peptide fragment.

8. The composition of claim 7, wherein the NABS is covalently grafted to the drug.

9. The composition of claim 1 further comprising a transfection agent.

10. The composition of claim 13 wherein the transfection agent is selected from a polyethylenimine, polyalkylenimine, cationic lipid, cationic peptide, polyamine, protamine, polyarginine, polylysine, polyamidoamine, or a conjugate thereof.

11. The composition of claim 1, wherein the nucleic acid carrier is conjugated to a masking agent.

12. The composition of claim 11, wherein the masking agent is selected from a lipid, vitamin, alkyl chain, fatty acid, polysaccharide, cyclodextrin, poly(ethylene)glycol, poly(alkylene)oxide, HPMA, protein, antibody, human serum protein, polyglutamate, polypeptide, oligopeptide, peptide fragment, amino acid, or a combination thereof

13. The composition of claim 1 further comprising a targeting moiety.

14. The composition of claim 13 wherein the targeting moiety is selected from a membrane transduction peptide, fusogenic peptide, membrane permeabilizing agent, antibody, or an antibody fragment.

15. The composition of claim 1 wherein the drug is selected from a protein drug, peptide drug, interferon, α-interferon, β-interferon, or an antibody drug.

16. The composition of claim 1 wherein the nucleic acid carrier is a nucleoside, nucleotide, nucleoside analogue, nucleoside conjugate, nucleoside polymer, or a nucleoside derivative thereof.

17. The composition of claim 1 wherein the nucleic acid carrier comprises a nucleic acid ligand that binds a drug.

18. A method of presenting a drug to a cell comprising the steps of 1) combining a nucleic acid carrier and a drug to form a nucleic acid carrier-drug complex and 2) administering said nucleic acid carrier-drug complex to a cell.

19. The method of claim 18, wherein the nucleic acid-drug complex is administered to an organism, mammal, or human patient.

20. The composition of claim 17 wherein the targeting moiety is conjugated to a transfection agent or a NABS.

21. The composition of claim 1 wherein the nucleic acid carrier is a nucleic acid.

22. The method of reducing the toxicity of a toxic substance in a patient comprising the steps of 1) administering a nucleic acid carrier to the patient and 2) forming a complex between the toxic substance and the nucleic acid carrier.

23. A drug masking, pharmaceutical composition, suitable for delivery of a therapeutic agent to a cell, the composition consisting of:
a) a dosage form of a composition comprising:
(i) a nucleic acid suitable for complexing with the therapeutic agent
(ii) a therapeutically effective amount of a therapeutic agent, said therapeutic agent suitable in forming a non-covalent complex with said nucleic acid,
wherein the ratio of said nucleic acid present in the composition is sufficient to sufficiently complex with said therapeutic agent, such that the percentage of the complexed therapeutic agent in the dosage form is between 40% and 100% complexed to the nucleic acid.
b) the nucleic acid is a modified nucleic acid.

24. The composition of claim 23, wherein said therapeutic agent is selected from mitoxantrone.
